(19) United States
(12) Patent Application Publication
(10) Pub. No.: US 2021/0293815 A1
(43) Pub. Date: Sep. 23, 2021

(54) SELF- ADMINISTRABLE DIAGNOSTIC TEST WITH INTEGRATED SWAB

(71) Applicant: Detect, Inc., Guilford, CT (US)

(72) Inventors: Jonathan M. Rothberg, Guilford, CT (US); Spencer Glantz, West Hartford, CT (US); Benjamin Rosenbluth, Hamden, CT (US); Owen Kaye-Kauderer, Brooklyn, NY (US)

(73) Assignee: Detect, Inc., Guilford, CT (US)

(21) Appl. No.: 17/067,594

(22) Filed: Oct. 9, 2020

Related U.S. Application Data
(60) Provisional application No. 63/074,524, filed on Sep. 4, 2020.
(60) Provisional application No. 63/027,859, filed on May 20, 2020.
(60) Provisional application No. 63/016,797, filed on Apr. 28, 2020.
(60) Provisional application No. 62/991,039, filed on Mar. 17, 2020.

(51) Int. Cl. G01N 33/569; G01N 33/52

(52) U.S. Cl. CPC G01N 33/56983; G01N 2333/165; G01N 33/521

(57) ABSTRACT

Provided herein, in some embodiments, are rapid diagnostic tests comprising a lateral flow strip and an integrated swab head. The tests may be used to detect, for example, one or more pathogens, such as viral, bacterial, fungal, parasitic, or protozoan pathogens. Further embodiments provide methods of detecting genetic abnormalities. Nucleic acid tests using the diagnostic tests are provided.

### CROSS-REFERENCE TO RELATED APPLICATIONS

The present application claims the benefit under 35 U.S.C. § 119(e) of U.S. Provisional Patent Application No. 62/991,039, filed Mar. 17, 2020 under Attorney Docket No. H0966.70014US00, titled “Viral Rapid Test,” U.S. Provisional Patent Application No. 63/016,797, filed Apr. 28, 2020 under Attorney Docket No. H0966.70014US05, titled “Sample Swab with Built-In Illness Test,” U.S. Provisional Patent Application No. 63/027,859, filed May 20, 2020 under Attorney Docket No. H0966.70014US15, titled “Rapid Self Administrable Test,” and U.S. Provisional Patent Application No. 63/074,524, filed Sep. 4, 2020 under Attorney Docket No. H0966.70014US09, titled “Rapid Diagnostic Test with Integrated Swab,” each of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Feb. 23, 2021, is named H096670035US00-SEQ and is 8 kilobytes in size.

### BACKGROUND

Viral infections, such as coronaviruses and influenzas, commonly cause mild or moderate respiratory tract infections in humans. However, in some humans, viral infections are fatal. Certain viruses, such as the coronavirus disease 2019 (COVID-19), have proven to be more fatal than other viral infections. The current lack of treatment or vaccine for the novel virus has resulted in a pandemic. The ongoing crisis associated with COVID-19 illustrates the importance of developing rapid testing methods, so that populations may be tested more efficiently and appropriate public health measures may be enacted.

### SUMMARY

According to some aspects, a diagnostic device for detecting a disease is provided, the diagnostic device comprising a housing, a lateral flow strip integrated with the housing including a lysis region comprising at least one lysis buffer, and an amplification region comprising one or more lyophilized amplification reagents, and a swab coupled to the lateral flow strip and arranged outside the housing.

According to some aspects, a system is provided for performing a self-administrable test to detect a disease, the system comprising a diagnostic device comprising a housing, a lateral flow strip integrated with the housing including a lysis region comprising at least one lysis buffer, and an amplification region comprising one or more lyophilized amplification reagents, and a swab coupled to the lateral flow strip and arranged outside the housing, and a container comprising a rehydration buffer, wherein the container is configured to be coupled to the diagnostic device such that the swab is arranged within the rehydration buffer.

According to some aspects, a method of detecting a pathogen is provided, the method comprising collecting a biological sample from a first user's body, wherein a second user manipulates a diagnostic device to collect the biological sample from the first user's body, the diagnostic device comprising a lateral flow strip including a lysis region comprising at least one lysis buffer and an amplification region comprising one or more lyophilized amplification reagents, and a swab coupled to the lateral flow strip, wherein the biological sample is collected on the swab, and performing, by the lateral flow strip of the diagnostic device lysing of at least some of the biological sample, amplification of nucleic acids from the biological sample, screening of the amplified nucleic acids for the pathogen, and producing a visual indication of whether or not the pathogen is present in the biological sample.

The foregoing apparatus and method embodiments may be implemented with any suitable combination of aspects, features, and acts described above or in further detail below. These and other aspects, embodiments, and features of the present teachings can be more fully understood from the following description in conjunction with the accompanying drawings.

### BRIEF DESCRIPTION OF DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIGS. 1A and 1B depict a cross-sectional view and an exterior view, respectively, of a diagnostic device for performing a self-administrable nucleic acid test, according to some embodiments;

FIGS. 2A-2B depict a system comprising a diagnostic device and a container containing a buffer, according to some embodiments;

FIG. 3 is a flowchart of a method of performing a diagnostic test for one or more pathogens using a self-administrable nucleic acid test, according to some embodiments;

FIG. 4 depicts a schematic of a portable computing device determining a detection result from a diagnostic device based on fiducial markers, according to some embodiments;

FIG. 5 depicts a cross-sectional view of a diagnostic device for performing a self-administrable nucleic acid test, according to some embodiments;

FIG. 6 depicts examples of dual haptens for labeling in a diagnostic device as described herein, according to some embodiments;

FIG. 7 depicts an illustrative lateral flow strip key for interpreting results, according to some embodiments;

FIG. 8A depicts a schematic illustrating a test laboratory workflow, according to some embodiments;

FIG. 8B illustrates a series of photographs of lateral flow tests showing test results following use of different concentrations of UDG and dUTP during processing in a test laboratory workflow, according to some embodiments; and

FIG. 9 depicts a block diagram of an illustrative computing device which may be suitable for performing certain embodiments described herein.

### DETAILED DESCRIPTION

Nucleic acid tests are techniques used to detect particular nucleic acid sequences and are often used to detect pathogens such as a virus. In contrast to some other tests, nucleic acid tests (sometimes called “NATs”) detect genetic material rather than antibodies or antigens. In many cases, NATs can provide earlier detection of a disease than antibody or antigen tests because a NAT directly detects the presence of the pathogen rather than the antibodies or antigens that may be produced in the bloodstream as a result of the pathogen.

There are typically several steps in a NAT, including a lysis step in which cells from a sample are broken down, and an amplification step in which the genetic material from the cells is amplified by making copies of it. NATs are often performed in the laboratory wherein the lysis step may be performed by mechanical means such as a sonicator, and amplification may include polymerase chain reaction (PCR) techniques in which the lysed sample is exposed to reactants in repeated cycles of heating and cooling. In some cases, a NAT may include pipetting of a lysed sample onto a lateral flow strip along which the sample flows, undergoing amplification reactions within the strip.

In each of these approaches, contamination must be controlled for—since the amount of genetic material being detected is generally quite small, even a small amount of contamination can produce a spurious result. In particular, contamination from amplicons, which can spread as aerosols in the laboratory environment, can easily lease to a false positive test for the genetic material of interest.

The inventors have recognized techniques for performing a nucleic acid test in which a sample collection swab and a lateral flow strip including lysis and amplification stages are integrated into a single device. At least the amplification portion of the lateral flow strip may be sealed within the device to protect against amplicon contamination. This configuration may allow for a self-administrable test for a pathogen using the device, wherein a user initially collects a sample from their body using the swab. The sample may be lysed and amplified by being drawn up the lateral flow strip, which is fluidically coupled to the swab and is at least partially housed inside the device to avoid contamination. The device may also include one or more test lines that detect the presence of a particular pathogen within the amplified material. As a result, the user may be able to collect a sample from their body using the device, then observe on the device whether or not the pathogen was detected in the sample. Alternatively, a health care professional (e.g., in a point of care setting) may collect a sample from a subject's body using the swab, followed by lysing and amplification of the sample within the lateral flow strip, etc. as described above.

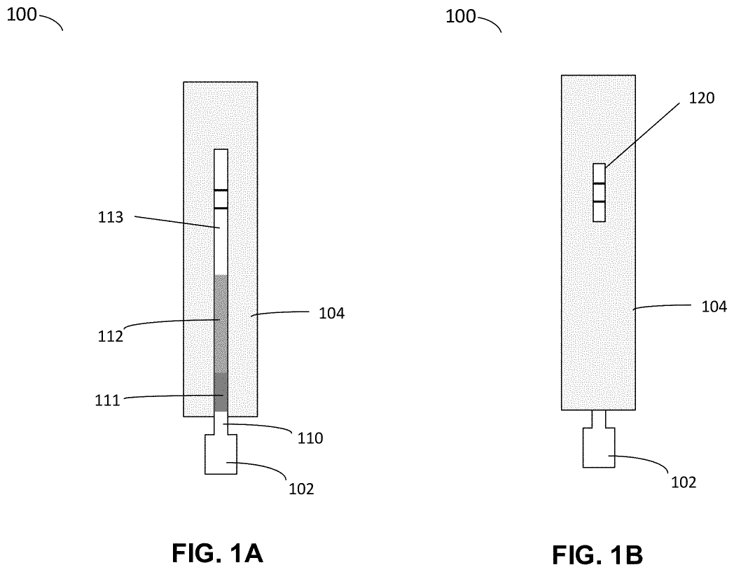

FIGS. 1A and 1B depict a cross-sectional view and an exterior view, respectively, of a diagnostic device for performing a self-administrable nucleic acid test, according to some embodiments. Device 100 comprises a swab 102, lateral flow strip 110 and housing 104. As may be seen from the cross-sectional view of FIG. 1A, the lateral flow strip is arranged at least partially within the housing 104, with the swab 102 arranged exterior to the housing. The lateral flow strip includes a lysis region 111, an amplification region 112 and a detection region 113. At least some of the detection region is visible through a transparent portion 120 of the housing 104.

In operation, a user may manipulate the device 100 to obtain a biological sample (e.g., from their own body) on the swab 102. Subsequently, biological material from the swab may be drawn along the lateral flow strip 110, resulting in lysis of the material in the lysis region 111 and amplification of lysed material in the amplification region 112. The amplified material may be drawn into the detection region 113 wherein detection of one or more pathogens is performed, resulting in a visible indication when a positive detection is made by the detection region. Such detection may be made by one or more test lines within the detection region 113. In some embodiments, the detection region may include one or more control lines to ensure that the device is functioning correctly and to provide context for any indications of positive or negative detection of a pathogen.

According to some embodiments, the housing 104 may comprise a plurality of parts sealed together around the lateral flow strip 110. For instance, the housing may comprise top and bottom rigid sections (e.g., plastic sections) joined together via a snapfit or pressfit seal and/or welded together (e.g., via ultrasonic welding). Such housing parts may, for instance, comprise injection molded plastic parts.

According to some embodiments, the housing 104 may comprise a thin film formed around the lateral flow strip. In some embodiments, the thin film may be a flexible packaging lamination (e.g., a thermoplastic) formed around the lateral flow strip via laser welding and/or thermal sealing.

According to some embodiments, the housing other than the transparent portion 120 of the housing may include any number of transparent and/or opaque regions. For instance, the transparent portion 120 may be a transparent window within an opaque housing (or opaque portion of the housing), or may be a transparent portion of a transparent housing. In the case of a transparent window within a rigid housing, the window may be formed from an in-mold label (e.g., a plastic window within an injection molded housing part), or may be separately assembled from the housing and attached to the housing with a soft seal (e.g., with an elastomer or polyurethane, etc.) or with a hard seal (e.g., a rigid thermal seal).

According to some embodiments, the diagnostic device 100 may comprise a semi-permeable film or membrane configured to allow gas to exit the housing while inhibiting or preventing gas from entering the housing. If the interior volume of the housing 104 is sufficiently small, without such a film or membrane the fluid on the lateral flow strip or swab may be prevented or inhibited from entering the housing because gas within the housing may not be able to escape and/or due to vapor lock. A semi-permeable film or membrane, or a valve, arranged in a suitable location, such as within the housing, may mitigate this issue. Alternatively, the housing may be large enough that fluid entering the housing does cause the pressure within the housing to increase, but not a sufficient amount to prevent fluid flow along the lateral flow strip.

According to some embodiments, the exterior of the housing may include one or more fiducial targets. As described further below, in some embodiments a detection result produced by the diagnostic device 100 may be determined by a portable computing device (e.g., a mobile phone or tablet). Fiducial targets disposed on the housing may assist with the portable computing device identifying the detection region 113 based on a known spatial relationship between the fiducial target(s) and the detection region (e.g., the entire detection region and/or one or more test lines within the detection region). Fiducial targets may include any computer-recognizable pattern, including QR codes, AR codes, or the like.

The swab 102 may comprise any suitable absorbent material(s) for collecting a biological sample. The swab may be configured to collect an oral sample and/or a nasal sample. The swab may, for instance, comprise cotton, filter paper, cellulose-based materials, polyurethane, polyester, rayon, nylon, microfiber, viscose, alginate, or combinations thereof. In some embodiments, the swab may comprise an absorbent portion (e.g., comprising the absorbent material) and a stem portion (e.g., a handle, an applicator). In some embodiments, the swab is a foam swab (e.g., the swab portion comprises a foam material) and/or a flocked swab (e.g., the swab portion comprises flocked fibers). A stem portion of the swab may be formed from any suitable material. In some embodiments, the stem portion comprises a thermoplastic material (e.g., a polystyrene, a polyethylene, a polypropylene, a polystyrene, an olefin), a metal (e.g., aluminum), wood, paper, or another type of material. In some embodiments, the stem portion of a swab comprises one or more markings and/or flanges. The markings and/or flanges may, in some instances, indicate the appropriate depth of insertion (e.g., into a nasal cavity) during sample collection.

According to some embodiments, the swab 102 may be integrated with or otherwise attached to the lateral flow strip in that the swab 102 and lateral flow strip 110 may share a common substrate. For instance, the swab 102 may be formed from an end of the lateral flow strip with absorbent material added. In some embodiments, the swab 102 may be a separate component from the lateral flow strip that is fluidically coupled to the lateral flow strip such that fluid absorbed by the swab is drawn up into the lateral flow strip.

According to some embodiments, the lateral flow strip 110 may comprise one or more fluid-transporting layers comprising one or more materials that allow fluid transport (e.g., via capillary action). Non-limiting examples of suitable materials include polyethersulfone, cellulose, polycarbonate, nitrocellulose, sintered polyethylene, glass fibers, or combinations thereof.

According to some embodiments, the lysis region 111 of the lateral flow strip may comprise a lysis buffer, in addition to lysis enzymes and detergents. In some embodiments, the lysis buffer is contained in a blister pack which may be punctured to release the buffer at the appropriate time into the lysis region 111 (techniques for which are described below). In some embodiments, the lysis region is a lysis lateral flow strip; that is, the sample proceeds through the lysis region through capillary action and interacts with lysis reagents impregnated in the flow strip. After the sample has been lysed, the lysed material moves via capillary action to the amplification region.

In some embodiments, the amplification region 112 comprises a blister pack comprising a dilution buffer that is released at an appropriate time (techniques for which are described below). In some embodiments, the user is instructed to puncture the dilution buffer blister pack at the appropriate time by a mobile application, as described herein. In some embodiments, the amplification region comprises an amplification lateral flow strip, and the lateral flow strip is impregnated with reagents for reverse transcription and recombinase polymerase amplification (RPA). In some embodiments, the amplification region comprises an amplification lateral flow strip, and the lateral flow strip is impregnated with reagents for loop-mediated isothermal amplification (LAMP). In some embodiments, the amplification region comprises a lateral flow strip and the lateral flow strip is impregnated with reagents for a suitable amplification procedure (e.g., isothermal amplification technologies described herein). The sample is slowed, if necessary, by materials in the lateral flow strip, in order to ensure proper and sufficient amplification so that the processed sample will be able to be analyzed. The processed sample then progresses to the detection region of the test, which comprises a lateral flow test strip.

In some embodiments, the amplification region 112 of lateral flow strip 110 and the detection region 113 of lateral flow strip 110 are regions of a single continuous lateral flow strip. In other embodiments, the amplification lateral flow strip and the results lateral flow strip are two separate flow strips arranged adjacent to one another. In some embodiments, the detection region 113 of the lateral flow strip comprises at least three lines, each comprising a unique set of antibodies in order to screen the sample. In some embodiments, the sample may be screened for a human control to confirm that the sample is present. In some embodiments, the sample may be screened for a test control to confirm the lateral flow test completed successfully. In some embodiments, the sample may be screened for at least one viral disease (e.g., COVID-19). In some embodiments, the detection region 113 of the lateral flow strip 110 further screens for a second, and optionally, third, disease. The second and third diseases may include diseases that may be confused for COVID-19, such as influenza type A and influenza type B. Other diseases or targets of interest are also possible, including any suitable nucleic acid target of interest including a target associated with a pathogen of viral, bacterial, fungal, parasitic, protozoan or other origin.

According to some embodiments, the diagnostic device 100 may include one or more heating elements (not shown in FIGS. 1A-1B). A heating element may be arranged proximate to one or more regions of the lateral flow strip to provide heating to that region during while reactions take place within the strip. For instance, some amplification reactions may require, or may be improved with, the material within the strip being raised above room temperature. A suitable heating element may include a resistive heater. In some embodiments, the heater may be a USB-powered heat source. The housing 104 may be thermally-insulated to ensure user safety. According to some embodiments, a heating element arranged within the housing 104 of diagnostic device 100 may be configured to heat one or more regions of the lateral flow strip to at least 35° C., 40° C., 45° C., 50° C., 55° C., 60° C., 65° C., 70° C., 75° C., 80° C., 85° C., 90° C., or 95° C.

The diagnostic device 100 may be configured to detect the presence or absence of any suitable target nucleic acid sequence (e.g., from any pathogen of interest). For instance, any suitable target nucleic acid sequence may be detected in detection region 113 subsequent to lysis and amplification of a sample by binding a probe within the detection region to the target nucleic acid sequence. Target nucleic acid sequences may be associated with a variety of diseases or disorders, numerous examples of which are described below.

In some embodiments, the device may be configured to screen for a nucleic acid encoding a SARS-CoV-2 nucleocapsid protein, which is associated with COVID-19. The device 100 may, in some embodiments, be used to diagnose at least one disease or disorder caused by a pathogen, as described below. In some embodiments, the diagnostic device 100 may be configured so that a user of the device can differentiate between one or more diseases or disorders (e.g., a lateral flow test comprises more than one test line).

In some embodiments, the detection region 113 of the lateral flow strip 110 may be configured to detect (e.g., via a first test line) SARS-CoV-2 and may be configured to detect (e.g., via a second test line) an influenza (e.g., Type A or Type B). In some embodiments, the detection region 113 of the lateral flow strip 110 may be configured to detect SARS-CoV-2, influenza Type A, and influenza Type B. In some embodiments, the detection region 113 of the lateral flow strip 110 may be configured to detect (e.g., via a first test line) SARS-CoV-2 and may be configured to detect (e.g., via a second test line) SARS-CoV-2 having a D614G mutation in its spike protein (see, e.g., Korber et al., 2020), and optionally, may also be configured to detect (e.g., via a third test line) influenza (e.g., Type A, Type B, or both). In some embodiments, the detection region 113 of the lateral flow strip 110 may be configured to detect the presence or absence of 1, 2, 3, 4, 5, 6, 7, 8, 9, 10, or more different target nucleic acid sequences. In further embodiments, the detection region 113 of the lateral flow strip 110 may differentiate between viral and bacterial infections.

In some embodiments, the diagnostic device 100 may be configured to diagnose or detect a viral pathogen, such as pathogens that cause respiratory illness, including, but not limited to, coronaviruses, influenza viruses, rhinoviruses, parainfluenza viruses (e.g., parainfluenza 1-4), enteroviruses, adenoviruses, respiratory syncytial viruses, and metapneumonviruses. Other viral pathogens that diagnostic device 100 may be configured to detect may include, but are not limited to, adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Hantavirus, Middle East Respiratory Coronavirus; Zika virus; Norovirus; Chikungunya virus; Banna virus; or combinations thereof.

In some embodiments, the diagnostic device 100 may be configured to diagnose one or more bacterial pathogens. The bacterium described herein can be a Gram-positive bacterium or a Gram-negative bacterium. Bacterial pathogens may include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, Yersinia pestis, or combinations thereof.

In some embodiments, the diagnostic device 100 may be configured to detect one or more fungal pathogens. Examples of such fungal pathogens may include, but are not limited to Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), Mucoromycotina (e.g., Mucor circinelloides, Rhizopus oryzae, Lichtheimia corymbifera), or combinations thereof.

In some embodiments, the diagnostic device 100 may be configured to detect one or more protozoan pathogens. Examples of such protozoan pathogens may include, but are not limited to, Entamoeba histolytica, Giardia lambila, Trichomonas vaginalis, Trypanosoma brucei, T. cruzi, Leishmania donovani, Balantidium coli, Toxoplasma gondii, Plasmodium spp., Babesia microti, or combinations thereof.

In some embodiments, the diagnostic device 100 may be configured to detect one or more parasitic pathogens. Examples of such parasitic pathogens may include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, Wuchereria bancrofti, or combinations thereof.

In some embodiments, the diagnostic device 100 may be configured to detect any number of pathogenic animal diseases, including, but not limited to, bovine rhinotracheitis virus, bovine herpesvirus, distemper, parainfluenza, canine adenovirus, rhinotracheitis virus, calicivirus, canine parvovirus, Borrelia burgdorferi (Lyme disease), Bordetella bronchiseptica (kennel cough), canine parainfluenza, leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesevirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, Pastuerella multocida, or combinations thereof.

In some embodiments, the diagnostic device 100 may be configured to test water or food for contaminants (e.g., for the presence of harmful bacteria). Bacterial contamination of food and water results in foodborne diseases, which contribute to approximately 128,000 hospitalizations and 3000 deaths annually in the United States (CDC, 2016). The tests described herein may be used to detect specific contaminants (toxins). In particular, bacterial toxins produced by Staphylococcus spp., Bacillus spp. and Clostridium spp. account for the majority of foodborne illnesses. By processing a potentially contaminated food or water sample down to nucleic acids and then using the methods or tests described herein, the diagnostic device 100 can produce an indication of whether or not the sample contained the bacterial toxin (e.g., is contaminated). In other embodiments, the diagnostic device 100 may be utilized during a food production process, to ensure food safety prior to consumption.

Thus, in some embodiments, the diagnostic device 100 may be configured to determine whether a food or water sample is contaminated, for example, by screening the sample for one or more bacterial toxins produced by Staphylococcus spp., Bacillus spp. and Clostridium spp. In some embodiments, the toxins are produced by Clostridium botulinum, C. perfringens, Staphylococcus aureus, Bacillus cereus, Shiga-toxin-producing Escherichia coli (STEC), and/or Vibrio parahemolyticus. Illustrative toxins include, but are not limited to aflatoxin, cholera toxin, diphtheria toxin, Salmonella toxin, Shiga toxin, Clostridium botulinum toxin, endotoxin, and mycotoxin. In other embodiments, test samples may include water, liquid extracts of air filters, soil samples, building materials (e.g., drywall, ceiling tiles, wall board, fabrics, wall paper, and floor coverings), environmental swabs, or any other sample.

In some embodiments, the diagnostic device 100 may be configured to perform soil analysis by examining toxins, as described above, from a soil sample. Further, the diagnostic device 100 may be configured to analyze ammonia- and methane-oxidizing bacteria, fungi or other biological elements of a soil sample. Such information can be useful, for example, in predicting agricultural yields and in guiding crop planting decisions.

In some embodiments, the diagnostic device 100 may be configured to perform a diagnostic for one or more cancers. Cancer cells have unique mutations found in tumor cells and absent in normal cells. For example, specific cancer neoantigens and/or tumor-associated antigens (TAA) may be screened for by using the diagnostic device 100. Examples of TAAs include, but are not limited to MelanA (MART-I), gplOO (Pmel 17), tyrosinase, TRP-I, TRP-2, MAGE-I, MAGE-3, BAGE, GAGE-I, GAGE-2, p15(58), CEA, RAGE, NY-ESO (LAGE), SCP-I, Hom/Me1-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY-CO-I, RCAS1, SDCCAG16, TA-90 (Mac-2 binding protein\cyclophilin C-associated protein), TAAL6, TAG72, TLP, TPS5, or combinations thereof. Neoantigens, in some embodiments, arise from tumor proteins (e.g., tumor-associated antigens), are also known in the art. In some embodiments, the neoantigen comprises a polypeptide comprising approximately 10, 15, 20, 25, 30, 35, 40, 45, 50, 75, 100, 150, 200, 250, 10-250, 50-250, 100-250, or 50-150 amino acids (or at least about 10, 15, 20, 25, 30, 35, 40, 45, 50, 75, 100, 150, 200, or 250 amino acids) that is identical to a sequence of amino acids within a tumor antigen or oncoprotein (e.g., Her2, E7, tyrosinase-related protein 2 (Trp2), Myc, Ras, or vascular endothelial growth factor (VEGF)). In some embodiments, the sample is a blood sample, such as a plasma sample.

In some embodiments, the diagnostic device 100 may be configured to examine a subject's predisposition to certain types of cancer based on specific genetic mutations. As an example, mutations in BRCA1 and/or BRCA2 may indicate that a subject is at an increased risk of breast cancer, as compared to a subject who does not have mutations in the BRCA 1 and/or BRCA2 genes. Other genetic mutations that may be screened according to the methods provided herein include, but are not limited to, BARD1, BRIP1, TP53, PTEN, MSH2, MLH1, MSH6, NF1, PMS1, PMS2, EPCAM, APC, RB1, MEN1, MEN2, and VHL. Further, determining a subject's genetic mutation may help guide treatment decisions, as certain cancer drugs are indicated for subjects having specific genetic variants of particular cancers. For example, azathioprine, 6-mercaptopurine, and thioguanine all have dosing guidelines based on a subjects TPMT genotype (see, e.g., The Pharmacogeneomics Knowledgebase, pharmgkb.org).

In some embodiments, the diagnostic device 100 may be configured to perform genetic testing. In some embodiments, the target nucleic acid sequence may be associated with a genetic disorder selected from the group consisting of: hemophilia, sickle cell anemia, α-thalassemia, β-thalassemia, Duchene muscular dystrophy (DMD), Huntington's disease, severe combined immunodeficiency, Marfan syndrome, hemochromatosis, and cystic fibrosis. In some embodiments, the target nucleic acid sequence may be a portion of nucleic acid from a genomic locus of at least one of the following genes: CFTR, FMR1, SMN1, ABCB 11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASP A, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CUT A, CLN3, CLN5, CLN6, CLN8, CLRN1, CNGB3, COL27A1, COL4A3, COL4A4, COL4A5, COL7A1, CPS1, CPT1A, CPT2, CRB 1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBAI, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAM A3, LAMB3, LAMC2, LCA5, LDLR, LDLRAP1, LHX3, LIFR, LIP A, LOXHD1, LPL, LRPPRC, MAN2B1, MCOLN1, MED 17, MESP2, MFSD8, MKS1, MLC1, MMAA, MMAB, MMACHC, MMADHC, MPI, MPL, MPV17, MTHFR, MTM1, MTRR, MTTP, MUT, MY07A, NAGLU, NAGS, NBN, NDRG1, NDUFAF5, NDUFS6, NEB, NPC1, NPC2, NPHS1, NPHS2, NR2E3, NTRK1, OAT, OP A3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

Returning to the lysis region 111 of the lateral flow strip 110, the lysis region may include any suitable reagents for performing lysis, including reagents suitable for performing lysis at room temperature and/or reagents for performing lysis above room temperature. As discussed above, the diagnostic device 100 may include a heater; in some embodiments a heater may be configured to heat the lysis region 111 and thereby cause lysis reactions to occur above room temperature.

According to some embodiments, the lysis region 111 may comprise one or more lyophilized lysis reagents. Such lyophilized lysis reagents, may include Thermolabile Uracil-DNA Glycosylase (UDG) (e.g., with a concentration of 0.02 U/uL) and/or murine RNAse inhibitor (e.g., with a concentration of 1 U/uL). The lyophilized lysis reagents may be, in some embodiments, shelf-stable for 1, 1.5, 2, 2.5, 3, 3.5, 4, 4.5, 5, or more years.

According to some embodiments, the lysis region 111 may be configured to perform lysis by applying heat to a sample (thermal lysis). In some embodiments, the diagnostic device 100 may comprise a heat source that may be operated to produce two different temperature zones that are both above room temperature. For instance, a heater configured to heat the lysis region 111 may be configured to produce a first temperature zone between 60° C.-90° C. and a second temperature zone between 30° C.-40° C., for example, temperature zones at 65° C. and 37° C.

In some embodiments, the lateral flow strip 110 may be configured to perform reverse transcription to convert viral RNA into DNA (a reverse transcription region is not shown in FIG. 1A but, if present, may be arranged between the lysis region 111 and amplification region 112). In some embodiments, the lateral flow strip 110 may include a reverse transcriptase and a DNA-dependent polymerase. Reverse transcriptases (also known as RNA-dependent DNA polymerases), are enzymes having a DNA polymerase activity that transcribe single-stranded RNA (ssRNA) into a complementary single stranded DNA (cDNA) by polymerizing deoxyribonucleotide triphosphates (dNTPs). In some embodiments, RNAse may be used to digest the RNA away from an RNA-DNA hybrid. RNAses are commercially available (e.g., from ThermoFisher Scientific, New England BioLabs, etc.).

Amplification region 112 may include any suitable technology to amplify the sample on the lateral flow strip 110. In some embodiments, the amplification technology is an isothermal amplification technology. Isothermal amplification technologies may include, but are not limited to, nucleic acid sequence-based amplification (NASBA), strand displacement amplification (SDA), nicking enzyme amplification reaction (NEAR), loop-mediated isothermal amplification (LAMP), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), whole genome amplification (WGA), and recombinase polymerase amplification (RPA). In some embodiments, the amplification region 112 may be configured to perform loop-mediated isothermal amplification (LAMP). In some embodiments, the amplification region 112 may be configured to perform recombinase polymerase amplification (RPA). In some embodiments, the amplification region 112 may be configured to perform NEAR. In some embodiments, the amplification region 112 may include a modified nucleotide, for example, deoxyuridine triphosphate (dUTP).

In some embodiments, the amplification region 112 may be configured to perform recombinase polymerase amplification (RPA) in order to amplify the resulting DNA. RPA is an isothermal amplification technique that allows for fast, portable and extremely sensitive nucleic acid detection. RPA is a quick reaction (results are typically generated within 10 minutes) and does not require extensive instrumentation and/or reagents, making it well-suited for point-of-care use in settings with minimal resources. Following amplification, RPA products generated by the reaction may flow into the detection region 113 of the lateral flow strip 110. Depending on whether or not the target nucleic acid was detected, visible colored lines may, or may not, form within the detection region of the strip.

In some embodiments, the amplification region 112 of the lateral flow strip 110 comprises a recombinase agent, which is contacted with a forward and a reverse nucleic acid primer to form a first and a second nucleoprotein primer. Illustrative primers for inclusion in the recombinase agent are listed in Table 1 below. The oligonucleotide primers and probes for amplification and detection of SARS-CoV-2 were selected from regions of the virus nucleocapsid (N) gene to maximize inclusivity across known SARS-CoV-2 strains and minimize cross-reactivity with related viruses and genomes likely to be present in the sample. In other embodiments, the oligonucleotide primers and probes are selected from regions of the virus' envelope (E) gene, membrane (M) gene, and/or spike (S) gene. The panel, in some embodiments, is designed for specific detection of the SARS-CoV-2 (one primer/probe set). An additional primer/probe set to detect the human RNase P gene (RP) in control samples and clinical specimens is also included in some embodiments.

In some embodiments, the amplification region 112 may be configured to perform RPA with a single-stranded DNA probe that comprises a 5′ initial hybridization region, an abasic site, a detection region downstream of the abasic site, and a 3′ blocking group. In some embodiments, the initial hybridization region is located toward the 5′ end of the probe and the detection region is located toward the 3′ end of the probe. In some embodiments, the probe comprises, from 5′ to 3′, the initial hybridization region, the abasic site, the detection region, and the 3′ blocking group.

The initial hybridization region of the RPA probe may be sufficiently complementary to the target nucleic acid sequence (e.g., a region of the DNA encoding for the SARS-CoV-2 nucleocapsid protein). “Sufficiently complementary” as used herein, refers to a degree of complementarity sufficient to facilitate RPA. The initial hybridization region may be 15-100 nucleotides (nt) in length, for example 15-90 nt, 15-80 nt, 15-70 nt, 15-60 nt, 15-50 nt, 15-40 nt, 15-30 nt, 15-20 nt, 20-100 nt, 20-90 nt, 20-80 nt, 20-70 nt, 20-60 nt, 20-50 nt, 20-40 nt, 20-30 nt, 30-100 nt, 30-90 nt, 30-80 nt, 30-70 nt, 30-60 nt, 30-50 nt, 30-40 nt, 40-100 nt, 40-90 nt, 40-80 nt, 40-70 nt, 40-60 nt, 40-50 nt, 50-100 nt, 50-90 nt, 50-80 nt, 50-70 nt, 50-60 nt, 60-100 nt, 60-90 nt, 60-80 nt, 60-70 nt, 70-100 nt, 70-90 nt, 70-80 nt, 80-100 nt, 80-90 nt, or 90-100 nt. In some embodiments, the initial hybridization region is 20-50 nt in length. In some embodiments, the initial hybridization region may be 10 nt, 15 nt, 18 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, 52 nt, 54 nt, 55 nt, 56 nt, 58 nt, 60 nt, or more base pairs in length.

In some embodiments, the initial hybridization region of the RPA probe may comprise a 5′ overhang when bound to its target nucleic acid sequence. The 5′ overhang may be 1-50 nucleotides (nt) in length. In some embodiments the 5′ overhang is 1-50 nt, 1-40 nt, 1-30 nt, 1-25 nt, 1-20 nt, 1-15 nt, 1-10 nt, 1-9 nt, 1-8 nt, 1-7 nt, 1-6 nt, 1-5 nt, 1-4 nt, 1-3 nt, 1-2 nt, 2-50 nt, 2-40 nt, 2-30 nt, 2-25 nt, 2-20 nt, 2-15 nt, 2-10 nt, 2-9 nt, 2-8 nt, 2-7 nt, 2-6 nt, 2-5 nt, 2-4 nt, 2-3 nt, 3-50 nt, 3-40 nt, 3-30 nt, 3-25 nt, 3-20 nt, 3-15 nt, 3-10 nt, 3-9 nt, 3-8 nt, 3-7 nt, 3-6 nt, 3-5 nt, 3-4 nt, 4-50 nt, 4-40 nt, 4-30 nt, 4-25 nt, 4-20 nt, 4-15 nt, 4-10 nt, 4-9 nt, 4-8 nt, 4-7 nt, 4-6 nt, 4-5 nt, 5-50 nt, 5-40 nt, 5-30 nt, 5-25 nt, 5-20 nt, 5-15 nt, 5-10 nt, 5-9 nt, 5-8 nt, 5-7 nt, 5-6 nt, 10-50 nt, 10-40 nt, 10-30 nt, 10-25 nt, 10-20 nt, 10-15 nt, 15-50 nt, 15-40 nt, 15-30 nt, 15-25 nt, 15-20 nt, 20-50 nt, 20-40 nt, 20-30 nt, 20-25 nt, 30-50 nt, 30-40 nt, 40-50 nt, or 45-50 nt. In some embodiments, the 5′ overhang is 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, or more nucleotides in length. In some embodiments, the initial hybridization region is blunt-ended (there is no overhang).

In some embodiments, the detection region of the RPA probe may be sufficiently complementary to the target nucleic acid sequence. In some embodiments, the detection region is 2-20 nt in length, for example, 2-18 nt, 2-16 nt, 2-15 nt, 2-14 nt, 2-13 nt, 2-12 nt, 2-10 nt, 2-9 nt, 2-8 nt, 2-7 nt, 2-6 nt, 2-5 nt, 2-4 nt, 2-3 nt, 3-18 nt, 3-16 nt, 3-15 nt, 3-14 nt, 3-13 nt, 3-12 nt, 3-10 nt, 3-9 nt, 3-8 nt, 3-7 nt, 3-6 nt, 3-5 nt, 3-4 nt, 4-18 nt, 4-16 nt, 4-15 nt, 4-14 nt, 4-13 nt, 4-12 nt, 4-10 nt, 4-9 nt, 4-8 nt, 4-7 nt, 4-6 nt, 4-5 nt, 5-18 nt, 5-16 nt, 5-15 nt, 5-14 nt, 5-13 nt, 5-12 nt, 5-10 nt, 5-9 nt, 5-8 nt, 5-7 nt, 5-6 nt, 6-18 nt, 6-16 nt, 6-15 nt, 6-14 nt, 6-13 nt, 6-12 nt, 6-10 nt, 6-9 nt, 6-8 nt, 6-7 nt, 7-20 nt, 7-18 nt, 7-16 nt, 7-15 nt, 7-14 nt, 7-13 nt, 7-12 nt, 7-10 nt, 7-9 nt, 7-8 nt, 8-20 nt, 8-18 nt, 8-16 nt, 8-15 nt, 8-14 nt, 8-13 nt, 8-12 nt, 8-10 nt, 8-9 nt, 9-20 nt, 9-18 nt, 9-16 nt, 9-15 nt, 9-14 nt, 9-13 nt, 9-12 nt, 9-10 nt, 10-20 nt, 10-18 nt, 10-16 nt, 10-15 nt, 10-14 nt, 10-13 nt, 10-12 nt, 11-20 nt, 11-18 nt, 11-16 nt, 11-15 nt, 11-14 nt, 11-13 nt, 11-12 nt, 12-20 nt, 12-18 nt, 12-16 nt 12-15 nt, 12-14 nt, 12-13 nt, 13-20 nt, 13-18 nt, 13-16 nt, 13-15 nt, 13-14 nt, 14-20 nt, 14-18 nt, 14-16 nt, 14-15 nt, 15-20 nt, 15-18 nt, 15-16 nt, 16-20 nt, 16-18 nt, 17-20 nt, 18-20 nt, of 19-20 nt In some embodiments, the detection region is 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, or longer. In some embodiments, the detection region is <15 nt.

In some embodiments, the detection region of the RPA probe may comprise a 3′ overhang (e.g., a single-stranded portion of the target nucleic acid sequence). The 3′ overhang may be 1-50 nucleotides (nt) in length. In some embodiments the 3′ overhang is 1-50 nt, 1-40 nt, 1-30 nt, 1-25 nt, 1-20 nt, 1-15 nt, 1-10 nt, 1-9 nt, 1-8 nt, 1-7 nt, 1-6 nt, 1-5 nt, 1-4 nt, 1-3 nt, 1-2 nt, 2-50 nt, 2-40 nt, 2-30 nt, 2-25 nt, 2-20 nt, 2-15 nt, 2-10 nt, 2-9 nt, 2-8 nt, 2-7 nt, 2-6 nt, 2-5 nt, 2-4 nt, 2-3 nt, 3-50 nt, 3-40 nt, 3-30 nt, 3-25 nt, 3-20 nt, 3-15 nt, 3-10 nt, 3-9 nt, 3-8 nt, 3-7 nt, 3-6 nt, 3-5 nt, 3-4 nt, 4-50 nt, 4-40 nt, 4-30 nt, 4-25 nt, 4-20 nt, 4-15 nt, 4-10 nt, 4-9 nt, 4-8 nt, 4-7 nt, 4-6 nt, 4-5 nt, 5-50 nt, 5-40 nt, 5-30 nt, 5-25 nt, 5-20 nt, 5-15 nt, 5-10 nt, 5-9 nt, 5-8 nt, 5-7 nt, 5-6 nt, 10-50 nt, 10-40 nt, 10-30 nt, 10-25 nt, 10-20 nt, 10-15 nt, 15-50 nt, 15-40 nt, 15-30 nt, 15-25 nt, 15-20 nt, 20-50 nt, 20-40 nt, 20-30 nt, 20-25 nt, 30-50 nt, 30-40 nt, 40-50 nt, or 45-50 nt. In some embodiments, the 3′ overhang is 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, or more nucleotides in length. In some embodiments, the detection region is blunt-ended (there is no overhang).

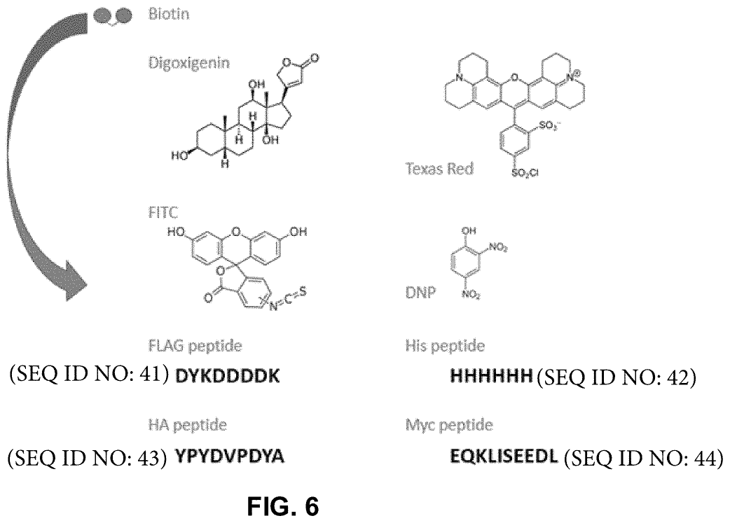

In some embodiments, the detection region of the RPA probe comprises at least two hapten labels, for example 2, 3, 4, 5, 6, 7, 8, 9, 10 or more hapten labels. Examples of haptens are provided in FIG. 6. In some embodiments, the hapten labels are synthesized as modified nucleotides. At least one hapten label, in some embodiments, is directly adjacent to the abasic site. In other embodiments, at least one hapten label is 1, 2, 3, 4, 5, 6, 7, 8, 9, 10, 11, 12, 13, or 14 nucleotides from the abasic site in the detection region. In some embodiments, at least one hapten label is appended to the 3′ end of the probe (e.g., the hapten label is a 3′ blocking group).

The 3′ blocking group of the RPA probe may prevent polymerase extension. In some embodiments, the 3′ blocking group may be used for lateral flow detection (e.g., the blocking group is a hapten, such as biotin-TEG).

Without wishing to be bound by theory, it is thought that the RPA probes described herein will permit a processed sample to be assayed directly; for example, loaded directly onto a lateral flow strip without requiring dilution. This is because the reaction results in the production of a small displaced tagged (labeled) oligonucleotides which freely diffuse from the RPA globule, while other components of the system stay trapped within the globule. The reaction occurs in the presence of 3K molecular weight PEG, and is therefore of low viscosity.

The probe binds its intended target in the sample, forming duplex DNA. The single-stranded probe is long enough (>15 bp) that it remains trapped within RPA globules. After the probe binds its target and forms a duplex, a DNA repair enzyme or structure-specific endonuclease or exonuclease creates a single base pair gap at the abasic site of the probe. Non-limiting examples of DNA repair enzymes include mutH, mutL, mutM, mutS, mutY, dam, thymidine DNA glycosylase (TDG), uracil DNA glycosylase, formamidopyrimidine DNA glycosylase, AlkA, MLH1, MSH2, MSH3, MSH6, FEN1 (RAD27), dnaQ (mutD), polC (dnaE), or combinations thereof. Examples of endonucleases that recognize an abasic (e.g., apurinic or apyrimidinic) site include, but are not limited to, APE 1 (or HAP 1 or Ref-1), Endonuclease III, Endonuclease IV, T4 endonuclease V, Endonuclease VIII, Fpg, and Hogg1. Examples of exonucleases include, but are not limited to, Exonuclease I, Exonuclease III Exonuclease V, RecJ exonuclease, Exonuclease T, Si nuclease, P1 nuclease, mung bean nuclease, T4 DNA polymerase, and CEL I nuclease.

Cleavage at the abasic site yields the duplexed 5′ initial hybridization region and a short (<15 bp) single-stranded oligonucleotide from the 3′ end (the detection region), which is removed from the duplex when strand displacing polymerases extend from the cleavage site. Illustrative polymerases include, but are not limited to, pol-α, pol-β, pol-δ, pol-δ, E. coli DNA polymerase I Klenow fragment, bacteriophage T4 gp43 DNA polymerase, Bacillus stearothermophilus polymerase I large fragment, Phi-29 DNA polymerase, T7 DNA polymerase, Bacillus subtilis Pol I, E. coli DNA polymerase I, E. coli DNA polymerase II, E. coli DNA polymerase III, E. coli DNA polymerase IV, E. coli DNA polymerase V and derivatives and combinations thereof.

The displaced oligonucleotide is able to diffuse from the RPA globule, owing to its relatively small size (<15 bp). The sample is then assayed and the presence or absence of the oligonucleotide is determined. For example, the sample may be loaded onto a lateral flow strip, where the low viscosity RPA product advances along the strip via capillary action. As described herein, the lateral flow strip comprises immobilized antibodies specific for at least one of the hapten labels on the oligonucleotide, so that the oligonucleotide, if present, will be captured in the correct location (e.g., first test line) on the lateral flow test. The oligonucleotide may then be visualized using a second labeled antibody against a second hapten present on the oligonucleotide (e.g., with a gold-conjugated second antibody).

In some embodiments, the amplification region 112 of the lateral flow strip 110 may be configured to perform a multiplexing approach to RPA. A combination of primers and probes may be included in the amplification region so that the resulting RPA product comprises oligonucleotides with unique hapten labels. In this way, the lateral flow strip may indicate the presence of one or more pathogens (e.g., SARS-Cov-2, influenza type A, influenza type B), a human RNA control, and a positive test control (e.g., non-targeting test control).

A conventional RPA process is also envisioned. The first and second nucleoprotein primers are incubated with a double stranded target sequence to form a first double stranded structure at a first portion of said first strand and form a double stranded structure at a second portion of said second strand so the 3′ ends of said first nucleic acid primer and said second nucleic acid primer are oriented towards each other on a given template DNA molecule. Next, the 3′ end of said first and second nucleoprotein primers are extended by DNA polymerases to generate first and second double stranded nucleic acids, and first and second displaced strands of nucleic acid. The steps are repeated until sufficient amplification product is generated. Unlike polymerase chain reaction (PCR) amplification, RPA does not require thermal melting of the double-stranded templates; that is, it is performed in an isothermal environment (e.g., 37° C.). Therefore, the tests may be performed at home or in an environment without access to traditional scientific or medical lab resources. In some embodiments, the enzymes are active at room temperature (e.g., 20° C.-22° C.), 37° C., or 65° C. In some embodiments, the enzymes are active at room temperature.

In some embodiments, the isothermal amplification is performed in a heat block at 37° C. In the process, recombinases form complexes with oligonucleotide primers and then catalyze the base-pairing of the oligonucleotide primer with a homologous stretch of duplex DNA, displacing one strand of the double-stranded DNA in the process. Single-stranded DNA binding proteins stabilize the resulting displaced strand. Only after an oligonucleotide primer has hybridized to its target sequence will polymerase initiate DNA amplification, ensuring specificity. In some embodiments, one of the two primer oligonucleotides carries a 5′ 6-FAM label.

Amplification is exponential, leading to the rapid accumulation of 6-FAM-labelled target DNA and the annealing of a biotinylated oligonucleotide probe. Only upon binding its intended target sequence will the probe be cleaved at an internal position by an endonuclease (e.g., an endonuclease type IV) in the reaction mix. This cleavage event in turn converts the probe into a priming site for polymerase extension. The amplicon generated by the cleaved biotinylated probe and the 5′ 6-FAM-labelled opposing amplification primer is dual-labeled. Within 10 minutes, a highly specific amplification process produces a dually-labeled amplicon. By including a second set of primers/probe, the isothermal amplification reaction is multiplexed to produce a distinctly labeled (DIG and Biotin) amplicon. The second set of primers/probe is targeted to a human RNA control. Additionally, dUTP (deoxyuridine-5′-triphosphate) is present in the mix of dNTPs used by the polymerase to generate the amplified product. As a result, all amplified products of the RPA reaction include some percentage of uracil in the nascent sequence. Included uracil molecules render any amplified product susceptible to UDG digestion/fragmentation employed in the initial step of each assay, preventing residual amplification products from generating false positive results in subsequent tests.

In some embodiments, the amplification region 112 of the lateral flow strip 110 comprises lyophilized amplification reagents. The lyophilized amplification reagents may comprise one or more of the following components: Reverse Transcriptase, murine RNAse inhibitor, T4 UvsX Protein, T4 UvsY Protein, T4 gp32 Protein, Endonuclease IV, Staphylococcus aureus DNA polymerase (Sau), Test Primer1 Fwd, Test Primer1 Rev Test Probe1 Control Primer1 Fwd, Control Primer1 Rev, Control Probe1, DL-Dithiothreitol, Phosphocreatine disodium hydrate, Creatine Kinase, Adenosine 5′-triphosphate disodium salt, Tris(hydroxymethyl)aminomethane (Tris), Deoxy-nucleotide triphosphates (dATP:dCTP:dGTP:dTTP), and Deoxyuridine triphosphate Solution (dU). In some embodiments, the lyophilized pellet comprises Reverse Transcriptase, murine RNAse inhibitor, T4 UvsX Protein, T4 UvsY Protein, T4 gp32 Protein, Endonuclease IV, Staphylococcus aureus DNA polymerase (Sau), Test Primer1 Fwd, Test Primer1 Rev Test Probe1 Control Primer1 Fwd, Control Primer1 Rev, Control Probe1, DL-Dithiothreitol, Phosphocreatine disodium hydrate, Creatine Kinase, Adenosine 5′-triphosphate disodium salt, Tris(hydroxymethyl)aminomethane (Tris), Deoxy-nucleotide triphosphates (dATP:dCTP:dGTP:dTTP), and Deoxyuridine triphosphate Solution (dU). For example, the lyophilized amplification reagents may comprise the following components:

| Component | Target Concentration |
|---|---|
| Reverse Transcriptase | 10 U/uL |
| murine RNAse inhibitor | 1 U/uL |
| T4 UvsX Protein | 0.12 mg/mL |
| T4 UvsY Protein | 0.06 mg/mL |
| T4 gp32 Protein | 0.6 mg/mL |
| Endonuclease IV | 0.0046 mg/mL |
| Staphylococcus aureus DNA polymerase (Sau) | 0.0128 mg/mL |
| Test Primer1 Fwd | 420 nM |
| Test Primer1 Rev | 420 nM |
| Test Probe1 | 120 nM |
| Control Primer1 Fwd | 420 nM |
| Control Primer1 Rev | 420 nM |
| Control Probe1 | 120 nM |
| DL-Dithiothreitol | 2 mM |
| Phosphocreatine disodium hydrate | 50 mM |
| Creatine Kinase | 0.1 mg/mL |
| Adenosine 5′-triphosphate disodium salt | 3 mM |
| Tris(hydroxymethyl)aminomethane (Tris) | 50 mM |
| Deoxy-nucleotide triphosphates | 0.2 mM each |
| (dATP:dCTP:dGTP:dTTP) | |
| Deoxyuridine triphosphate Solution (dU) | 0.2 mM |

Illustrative RPA primers are listed in Table 1 shown below. The primers and probes in Table 1 were designed to incorporate all COVID-19 variants, with a 99% threshold. Mismatches more than 3 bp away from the 3′ terminus of the primer were found to be well tolerated in RPA; however, if there are multiple mismatches within 3 bp of the 3′ terminus, this may inhibit the reaction completely. Therefore, in some embodiments, the primer has at least one mismatch at least 3, 4, 5, 6, 7, 8, 9, 10, or more bp away from the 3′ terminus of the primer. In some embodiments, the primer comprises 1, 2, 3, 4, 5, 6, 7, 8, 9, 10, or more mismatches. In some embodiments, the primer comprises 1 mismatch. In some embodiments, the primer has one mismatch within 3 bp of its 3′ terminus. In some embodiments, the primer does not have a mismatch within 3 bp of its 3′ terminus. The primers, in some embodiments, comprise 18, 19, 20, 21, 22, 23, 24, 25, 26, 27, 28, 29, 30, or more base pairs. In some embodiments, the enzymes used in the amplification (e.g., RPA) do not have any 3′ exonuclease activity (i.e., the enzymes cannot remove the 3′ end of the primer).

The forward primers, in some embodiments, comprise SEQ ID NOs: 1, 3, 5, 7, 9, or 11. The reverse primers, in some embodiments, comprise SEQ ID NOs: 2, 4, 6, 8, 10, or 12. In some embodiments the primer pairs comprise SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, or SEQ ID NO: 11 and SEQ ID NO: 12. In some embodiments, the primer pairs are 70%, 75%, 80%, 85%, 90%, 95%, or 100% identical to any one of SEQ ID NOs: 1-12. In some embodiments, the primer pairs comprise variants of any one or more of SEQ ID NOs: 1-12, wherein the variants are 1, 2, 3, 4, 5, or more base pairs longer or shorter than any one of SEQ ID NOs: 1-12. In some embodiments, the primer pair further comprises an antigenic tag (e.g., on the forward primer, the reverse primer, or both the forward and the reverse primers).

In some embodiments, the primer set further comprises a probe. The probe may comprise, for example, SEQ ID NOs: 13-16, or a sequence 70%, 75%, 80%, 85%, 90%, 95%, or 100% identical to any one of SEQ ID NOs: 13-16. In some embodiments, the primer set comprises SEQ ID NO: 9, SEQ ID NO: 10, and SEQ ID NO: 13. In some embodiments, the primer set comprises SEQ ID NO: 9, SEQ ID NO: 10, and SEQ ID NO: 14. In some embodiments, the primer set comprises SEQ ID NO: 11, SEQ ID NO: 12, and SEQ ID NO: 15. In some embodiments, the primer set comprises SEQ ID NO: 11, SEQ ID NO: 12, and SEQ ID NO: 16.

Set 1 and Set 2 primers (e.g., SEQ ID NOs: 9-12) were designed using the Nfo probes listed below (SEQ ID NOs: 13-16) and the BLASTn database (Altschul et al., Nucleic Acids Res. 25:3389-3402.). For Set 1, the amplicon size was 319 base pairs (bp). The forward primer (SEQ ID NO: 9) has at least a 4 bp mismatch sequence at the 3′ terminus, and a maximum background complementarity of 16/32 (50%). The reverse primer (SEQ ID NO: 10) also has at least a 4 bp mismatch sequence at the 3′ terminus, and a maximum background complementarity of 16/32 (50%). The Set 1 probes have less than 50% complementary to all background sequences, and no complementarity to SARS and cleavage sites. There are no sites similar to the human genome. For Set 2, the amplicon size was 332 base pairs (bp). The forward primer (SEQ ID NO: 11) has at minimum string of 3 mismatches from the 3′ terminus (except relative to Rothia mucilaginosa). The reverse primer (SEQ ID NO: 12) has no complementarity at the 3′ terminus sites for at least 5 bp-15 bp (depending on species). The Set 2 probes have complementarity to SARS between 4-30 nucleotides with 2 mismatches. In some embodiments, the cleavage site is positioned outside of the SARS complementarity region (e.g., the probe will only be cleaved and extended when bound to the COVID-19 target). There is some complementarity to between the human genome and the probes; however, the forward primer features 3 sequential mismatches at the 3′ terminus, so no amplification will occur.

TABLE 1
Illustrative Recombination Polymerase Amplification Primers

| | | SEQ |
|---|---|---|
| | | ID |
| RPA_primer | Sequence | NO: |
| RPA_fwd_1 | TCTGATAATGGACCCCAAAATCAGCGAAAT | 1 |
| RPA_rev_1 | CTCCATTCTGGTTACTGCCAGTTGAATCTG | 2 |
| RPA_fwd_3 | GCAACTGAGGGAGCCTTGAATACACCAAAA | 3 |
| RPA_rev_3 | TGAGGAAGTTGTAGCACGATTGCAGCATTG | 4 |
| RPA_fwd_2 | AAGGAACTGATTACAAACATTGGCCGCAAA | 5 |
| RPA_rev_2 | TTCCATGCCAATGCGCGACATTCCGAAGAA | 6 |
| RPA_fwd_4 | AAATTTTGGGGACCAGGAACTAATCAGACA | 7 |
| RPA_rev_4 | TGGCACCTGTGTAGGTCAACCACGTTCCCG | 8 |
| Set1_fwd | ACCCCAAAATCAGCGAAATGCACCCCGCATTA | 9 |
| Set1_rev | GTAGAAATACCATCTTGGACTGAGA | 10 |
| Set2_fwd | GTCTGATAATGGACCCCAAAATCAGCGA | 11 |
| Set2_rev | TAGTAGAAATACCATCTTGGACTGAGATCTTT | 12 |
| Set1_probe | AGAATGGAGAACGCAGTGGGGCGCGATCAAAACAACGTCGGC | 13 |
| | CCC | |
| Set1_probe_v1 | AGAATGGAGAACGCAGTGGGGCGCGATCA[dSpacer]AACAACGT | 14, 45 |
| | CGGCCCC[Block] | |
| Set2_probe | CAGTAACCAGAATGGAGAACGCAGTGGGGCGCGATCAAAACA | 15 |
| | ACGTCGGC | |
| Set2_probe_v | CAGTAACCAGAATGGAGAACGCAGTGGGGCGCGATCA[dSpacer] | 16, 46 |
| | AACAACGTCGGCC[Block] | |
| Forward | GTACTGCCACTAAAGCATACAATGTAACAC | 17 |
| Primer | | |
| Reverse | {6-FAM}AATATGCTTATTCAGCAAAATGACTTGATCT | 18 |
| Primer | | |
| Probe | {biotin}CAGACAAGGAACTGATTACAAACATTGGCCGCA{dSpacer} | 19, 47 |
| | ATTGCACAATTTGCC{phos} | |

In some embodiments, a modified RPA protocol is used. For example, RPA may be performed using small labels (e.g., labeled haptens) for the undiluted (direct) detection of RPA products on lateral flow strips (Powell et al., Analytical Biochem., 2018, 543(15): 108-115). As RPA reactions typically present as a phase-separated system, in which the core RPA proteins are found in colloidal globules, sometimes resulting in the sequestration of signals (e.g., biotin) and resulting in poor signaling results. To avoid this, smaller analytes (e.g., haptens) may be employed in conjunction with formamidopyrimidine DNA glycosylase (Fpg). In some embodiments, dual hapten probes are used. In some embodiments, the dual hapten probes comprise two haptens joined by a linker, such as a lysine residue. The smaller probes (e.g., dual hapten probes) are separated from the reaction until after amplification has occurred. Then, an Fpg probe against a specific amplicon target conjugated to the dual-hapten label is released. The probe binds to the specific amplicon target, and Fpg cleaves the dual-hapten label. Since the dual-hapten label is relatively small, it is able to readily leave the RPA globules. Then, the label may be detected by any means known in the art, such as with a sandwich immunoassay on a lateral flow strip (e.g., the lateral flow test comprises an antibody against one of the analytes and gold particles comprising antibodies against the second analyte). Examples of dual haptens for labeling are shown in FIG. 6 and include, biotin in combination with digoxigenin, FITC, Texas Red, dinitrophenol (DNP), FLAG peptide (DYKDDDDK; SEQ ID NO: 41), His peptide (HHHHHH; SEQ ID NO: 42), HA peptide (YPYDVPDYA; SEQ ID NO: 43), and Myc peptide (EQKLISEEDL; SEQ ID NO: 44).

In some embodiments, the amplification region 112 may be configured to perform loop-mediated isothermal amplification (LAMP) in order to amplify DNA. LAMP is a method of amplifying a target nucleic acid using at least four primers through the creation of a series of stem-loop structures. The LAMP technique is a single step amplification strategy, using a DNA polymerase having strand displacement activity, as described below. The reaction is isothermal and synthesis occurs rapidly once it begins. The technique is also highly specific for the target sequence, owing to its use of multiple primers.

In some embodiments, the amplification region 112 of the lateral flow strip 110 may comprise a plurality of primers for performing the LAMP protocol. These primers may include at least four primers which may be specific for eight regions within a target nucleic acid sequence, although in some cases two additional primers may be included. The primers within the amplification region 112 comprise at least a forward outer (upstream) primer (F3), a backward outer (downstream) primer (B3), a forward inner (upstream) primer (FIP), and a backward inner (downstream) primer (BIP). These primers correspond to six specific regions of the target gene: F3, F2, F1, B1c, B2c and B3c and six corresponding target gene complementary sequences: B3, B2, B1, F1c, F2c and F3c. The F3 sequence in the F3 primer can be complementary to the F3c sequence, and the B3 sequence in the B3 primer can be complementary to the B3c sequence. The F1c and F2 sequences in the FIP primer are complementary to the F1 and F2c sequences, respectively, and the B1c and B2 sequences in the BIP primer are respectively The B1 and B2c sequences are complementary. In some embodiments, a forward loop primer (Loop F or LF), and/or a backward loop primer (Loop B or LB) may also be utilized. The two loop primers are targeted to the cyclic structure formed during the amplification procedure, as described below, and can be used to accelerate the amplification. The target regions of the loop primer LF and the loop primer LB are between F1c-F2c and B1c-B2c, respectively.

As described herein, the amplification reaction performed by amplification region 112 when it is configured to apply the LAMP protocol produces a stem-loop DNA with inverted repeats of the target nucleic acid sequence. In some embodiments, LAMP is used to amplify RNA sequences. In such embodiments, reverse transcriptase (RT) is added to the reaction as described above. This type of LAMP amplification is referred to as RT-LAMP.

During LAMP, the BIP primer and reverse transcriptase initiate amplification by binding to a target sequence on the 3′ end of the RNA template and synthesizing a copy DNA strand. Next, the B3 primer binds to 3′ end of the RNA template strand as well, and together with DNA polymerase, simultaneously creates a new cDNA strand while displacing the previously made copy DNA strand.

The single-stranded copy forms a loop at its 3′ end by binding to itself. The FIP primer binds to the 5′ end of the single-strand copy and synthesizes a complementary strand with DNA polymerase. The F3 primer, with DNA polymerase, binds to the 5′ end of the single-stranded copy and synthesizes a new double-stranded DNA molecule while displacing the previously made single-stranded copy.

The newly-released single-stranded copy forms the foundation of the LAMP cycling amplification. The two ends of the DNA each fold in on themselves (self-annealing), yielding a DNA molecule having a dumbbell-like structure. This structure becomes a stem-loop when the FIP or BIP primer initiates DNA synthesis at one of the specific target sequence locations. This cycle can be started from either the forward or backward side of the dumbbell-shaped strand using the appropriate primer (FIP or BIP). Once amplification cycling begins, the strand undergoes self-primed DNA synthesis during the elongation stage of the amplification process.

In some embodiments, the LAMP primers may be designed by alignment and identification of conserved sequences in the target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In some embodiments, the target pathogen is SARS-CoV-2. LAMP primers for the virus are publicly available and known in the art (see, e.g., Tholoth et al., 2020 and Rabe et al., 2020). In some embodiments, the set of SARS-CoV-2 primers includes six primers each respectively having a sequence at least 90%, at least 95%, at least 96%, at least 97%, at least 98% or at least 99% identical to the primers provided in Table 2 below.

TABLE 2
Illustrative LAMP Primers (SARS-CoV-2)

| | | SEQ ID |
|---|---|---|
| Primer | Sequence (5′ to 3′) | NO: |
| F3_Set1 | CGGTGGACAAATTGTCAC | 20 |
| B3_Set1 | CTTCTCTGGATTTAACACACTT | 21 |
| Loop F_Set1 | TTACAAGCTTAAAGAATGTCTGAACACT | 22 |
| Loop B_Set1 | TTGAATTTAGGTGAAACATTTGTCACG | 23 |
| FIP1_Set1 | TCAGCACACAAAGCCAAAAATTTATCTGTGCAAAGGAA | 24 |
| | ATTAAGGAG | |
| BIP2_Set1 | TATTGGTGGAGCTAAACTTAAAGCCCTGTACAATCCCTT | 25 |
| | TGAGTG | |
| FIP2_Set1 | TCAGCACACAAAGCCAAAAATTTATTTTTCTGTGCAAAG | 26 |
| | GAAATTAAGGAG | |
| BIP2_Set1 | TATTGGTGGAGCTAAACTTAAAGCCTTTTCTGTACAATC | 27 |
| | CCTTTGAGTG | |
| F3_Set2 | TGCTTCAGTCAGCTGATG | 28 |
| B3_Set2 | TTAAATTGTCATCTTCGTCCTT | 29 |
| FIP_Set2 | TCAGTACTAGTGCCTGTGCCCACAATCGTTTTTAAACGGGT | 30 |
| BIP_Set2 | TCGTATACAGGGCTTTTGACATCTATCTTGGAAGCGACAACAA | 31 |
| Loop F_Set2 | CTGCACTTACACCGCAA | 32 |
| Loop B_Set2 | GTAGCTGGTTTTGCTAAATTCC | 33 |

In some embodiments, the diagnostic tests described herein further comprise a human positive control (e.g., RNase P). RNase P-specific LAMP primers are publicly available and known in the art (see, e.g., Curtis et al., J of Virol Methods, 2018, 255:91-97). In some embodiments, the set of RNase P primers includes six primers each respectively having a sequence at least 90%, at least 95%, at least 96%, at least 97%, at least 98% or at least 99% identical to the primers provided in Table 3 below.

TABLE 3
Illustrative RNase P Primers

| | | SEQ ID |
|---|---|---|
| Primer | Sequence (5′ to 3′) | NO: |
| F3 | TTGATGAGCTGGAGCCA | 34 |
| B3 | CACCCTCAATGCAGAGTC | 35 |
| FTP | GTGTGACCCTGAAGACTCGGTTTTAGCCACTGACT | 36 |
| | CGGATC | |
| BIP | CCTCCGTGATATGGCTCTTCGTTTTTTTCTTACAT | 37 |
| | GGCTCTGGTC | |
| Loop F | HEX-ATGTGGATGGCTGAGTTGTT | 38 |
| Loop B | CATGCTGAGTACTGGACCTC | 39 |
| Quencher | CAGCCATCCACAT-BHQ1 | 40 |

In some embodiments, the RT-LAMP reaction is performed within the amplification region 112 of lateral flow strip 110 at between 60° C. and 70° C., such as in the range 61° C.-69° C., 62° C.-68° C., 63° C.-67° C., or 64° C.-66° C. In some embodiments, the RT-LAMP reaction is performed at about 60° C., 61° C., 62° C., 63° C., 64° C., 65° C., 66° C., 67° C., 68° C., 69° C., or 70° C. In some embodiments, the RT-LAMP reaction is performed at 65° C.

In some embodiments, the RT-LAMP reaction is allowed to proceed for about 15 to about 45 minutes, such as about 20 minutes to about 40 minutes, or about 25 minutes to about 35 minutes. In some embodiments, the RT-LAMP reaction is allowed to proceed for about 30 minutes. In some embodiments, the RT-LAMP reaction is allowed to proceed for no more than 20 minutes, no more than 25 minutes, no more than 30 minutes, no more than 35 minutes, no more than 40 minutes or no more than 45 minutes.

The DNA polymerase used in the RT-LAMP reaction can be any enzyme appropriate for the assay, which can be selected by one of skill in the art. In some embodiments, the RT-LAMP reaction includes a DNA polymerase with high strand displacement activity. In some embodiments, the DNA polymerase is a DNA polymerase long fragment (LF) of a thermophilic bacteria such as Bacillus stearothermophilus (B st). Bacillus Smithii (Bsm), Geobacillus sp. M (GspM) or Thermodesulfatator indicus (Tin), an engineered variant therefrom or a Taq DNA polymerase variant. In some examples, the DNA polymerase is Bst LF DNA polymerase, GspM LF DNA polymerase, GspSSD LF DNA polymerase, Tin exo-LF DNA polymerase or SD DNA polymerase.

In some embodiments, the primers are labeled, e.g., biotin/FITC or biotin/digoxigenin (DIG). In this way, the resulting amplicons also carry the labels and can be detected using any of the methods described herein (e.g., lateral flow strip or colorimetric assay). In some embodiments, the fluorescent labels (e.g., FITC) may be quenched by a quencher or quenching strand that prevents it from signaling until the detection phase.

In some embodiments, the amplification region 112 may be configured to perform a nicking enzyme amplification reaction (NEAR) in order to amplify DNA. NEAR allows for isothermal unwinding and subsequent amplification of very small amplicons using a set of target-specific templates (primers), a nicking endonuclease, and a strand displacing DNA polymerase. Optionally, the NEAR method can include a probe, for example, a SARS-CoV-2 specific probe. The amplification region 112 of the lateral flow strip 110 may comprise a NEAR amplification composition that includes any one or more of these target-specific templates (primers), nicking endonucleases, strand displacing DNA polymerases, and/or probes.

In some embodiments, the amplification region 112 of the lateral flow strip 110 may comprise a first forward template that comprises a nucleic acid sequence having a hybridization region at the 3′ end that is complementary to the 3′ end of a target antisense strand (e.g., an antisense sequence to the reverse-transcribed SARS-CoV-2 nucleocapsid sequence), a nicking enzyme binding site and a nicking site upstream of the hybridization region, and a stabilizing region upstream of the nicking site. The first reverse template comprises a nucleic acid sequence having a hybridization region at the 3′ end that is complementary to the 3′ end of a target gene sense strand (e.g., a SARS-CoV-2 nucleocapsid gene sense strand), a nicking enzyme binding site and a nicking site upstream of the hybridization region, and a stabilizing region upstream of the nicking site. Designs of templates suitable for NEAR methods disclosed herein are provided in, for example, U.S. Pat. Nos. 9,617,586 and 9,689,031, each of which is incorporated herein by reference in its entirety.

In some embodiments, the amplification region 112 of the lateral flow strip 110 may comprise a NEAR amplification composition that includes a probe oligonucleotide, wherein the probe comprises a nucleotide sequence complementary to the target gene nucleotide sequence. The NEAR amplification composition may further comprise a DNA polymerase, at least one nicking enzyme, dNTPs, ddNTPs, or a combination thereof. In some embodiments, the NEAR composition further comprises dNTPs.

In some embodiments, the DNA polymerase is selected from the group consisting of Geobacillus bogazici DNA polymerase, Bst (large fragment), exo-DNA Polymerase, Manta 1.0 DNA Polymerase (Enzymatics 3 e).

In some embodiments, the one or more nicking enzymes of the NEAR amplification composition are selected from the group consisting of Nt. BspQI, Nb. BbvCi, Nb. BsmI, Nb. BsrDI, Nb. BtsI, Nt. AlwI, Nt. BbvCI, Nt. BstNBI, Nt. CviPII, Nb. Bpul OI, Nt. BpulOI and N. BspD61.

In some embodiments, the probe of the NEAR amplification composition is conjugated to a detectable label. In some embodiments, the detectable label is selected from the group consisting of a fluorophore, an enzyme, a quencher, an enzyme inhibitor, a radioactive label, a member of a binding pair, and a combination thereof. In some embodiments, one or more of the first forward template, the first reverse template comprises a least one modified nucleotide, spacer, or blocking group. In some embodiments, at least one modified nucleotide includes a 2′ modification. In some embodiments, amplification is performed under essentially isothermal conditions.

Returning to FIGS. 1A-B, once a biological sample collected by the swab 102 has been lysed and amplified by regions 111 and 112, respectively, of the lateral flow strip 110, the processed sample flows onto the detection region 113, which comprises at least one test line configured to detect one or more pathogens. Optionally, the detection region 113 may also include one or more control lines. The test lines configured to detect one or more pathogens may be configured to detect any of the pathogens indicated above by detecting the presence or absence of a relevant target nucleic acid sequence (or sequences). Any control lines present in the detection region 113 may be configured to determine whether the lateral flow strip test was successful (e.g., may detect moisture), and/or may be configured to determine whether a proper sample was provided. As an example of the latter, for a diagnostic device configured to detect a pathogen in a human subject, the control line may detect the presence or absence of a nucleic acid sequence expected to be present in all human samples.

According to some embodiments, the detection region 113 may comprise a conjugate pad comprising a dye. When products in the processed sample flow through the detection region 113 through passive capillary flow, they may flow over the conjugate pad, causing the dye to become attached to the products. This dye may subsequently be made visible in one or more of the test lines and/or control lines as a result of a product with the dye attached becoming captured by one or more reagents within the test line or control line.

According to some embodiments, the one or more test lines and/or control lines in the detection region 113 may comprise a band of immobilized antibodies. These antibodies may be configured to capture an amplified product with high specificity. By selecting appropriate antibodies, products for testing may be captured on one band whereas products for control may be captured on another band. When the products are captured on their respective bands, the dye attached to each product may generate a colored line on the lateral flow strip. The presence of a visible positive control band indicates that the test ran successfully, while the presence of the test band indicates the target analyte was detected in the patient's sample.

The lateral flow strip assay relies on capillary flow of the amplified sample through the nitrocellulose membrane and across discrete bands of capture antibodies. If the sample does not wick completely through the strip, then the assay is invalid. In some embodiments, a test line at the very end of the strip serves as a lateral flow control band. Upon proper lateral flow and wetting of the strip, this lateral flow control (LFC) band is rendered visible, independent of the detection of any target nucleic acid sequence by a test line of the detection region. Failure to detect LFC in any clinical specimen indicates failure of the sample to be properly delivered to the lateral flow strip, and an invalid result.

According to some embodiments, the detection region 113 comprises one or more primers configured to amplify a human or animal nucleic acid that is not associated with a target nucleic acid sequence from a pathogen, a cancer cell, a contaminant, etc. In some such embodiments, the human or animal nucleic acid may act as a control and is may be referred to herein as a “control nucleic acid”. For example, successful amplification and detection of a control nucleic acid may indicate that a sample was properly collected, and the diagnostic test was properly run (e.g., an amplification reaction was successful).

In some embodiments, a control nucleic acid may include a gene or portion of a gene that is widely expressed and/or expressed at a high level in a control organism (e.g., a human or other mammal). Such genes are typically referred to as “housekeeping genes”. Examples of housekeeping genes include but are not limited to GAPDH, B2M, ACTB, POLR2A, UBC, PPIA, HPRT1, GUSB, TBP, and H3F3A. In some embodiments, a control nucleic acid encodes a gene (or portion of a gene) selected from GAPDH, B2M, ACTB, POLR2A, UBC, PPIA, HPRT1, GUSB, TBP, H3F3A, POLR2A, RPLPO, L19, B2M, RPS17, ALAS1, CD74, RNase P, CK18, HMBS, IPO8, PGK1, and YWHAZ.

In some embodiments, the detection region 113 may comprise one or more pairs of electrodes for quantitative detection. The electrodes may be positioned on either side of the test lines, and conductive microspheres (e.g., attached to the amplicon of interest) are used for detection. With increased binding (e.g., increased density of conductive microspheres), conductivity across the test line increases. In some embodiments, when the conductivity reaches a threshold, a signal (e.g., LED light) is produced at each band to indicate that the target sequence is present.

In some embodiments, the detection region 113 is configured to perform a CRISPR/Cas analysis, as described in more detail below. For example, a guide RNA (gRNA) within the detection region 113 is designed to recognize a target nucleic acid sequence (e.g., a SARS-CoV-2-specific sequence), and once it finds the sequence, it activates a programmable nuclease within the detection region 113 (e.g., a Cas protein), which then cleaves a target molecule, releasing a detectable signal (e.g., a reporter molecule tagged with specific antibodies for the lateral flow test). In this way, the target nucleic acid sequence is indirectly detected via the reporter molecule. In some embodiments, the programmable nuclease is a Cas protein, such as Cas12a, Cas13, and Cas14.

In some embodiments, the detection region 113 and amplification region 112 are configured to perform a CRISPR/Cas detection process combined with an isothermal amplification technique to create a single step reaction (Joung et al., “Point-of-care testing for COVID-19 using SHERLOCK diagnostics,” 2020). For example, the amplification and CRISPR detection may be performed using reagents having compatible chemistries (e.g., reagents that do not interact detrimentally with one another and are sufficiently active to perform amplification and detection). In some embodiments, the isothermal amplification technique is LAMP, as described herein.

CRISPR/Cas detection platforms are known in the art. Examples of such platforms include SHERLOCK® and DETECTR® (see, e.g. Kellner et al., Nature Protocols, 2019, 14: 2986-3012; Broughton et al., Nature Biotechnology, 2020; Joung et al., 2020).

In some embodiments, the detection region 113 is configured to perform a CRISPR/Cas analysis to detect a target nucleic acid sequence (e.g., from a pathogen). Following amplification, the target nucleic acid sequences, if present, are detected using a guide RNA (gRNA) designed to recognize a specific target sequence (e.g., a SARS-CoV-2-specific sequence). If the sample comprises the target sequence, the gRNA will bind the target sequence and activate a programmable nuclease (e.g., a Cas protein, such as Cas12a, Cas13, or Cas14), which then cleaves a reporter molecule, releasing a detectable signal (e.g., a reporter molecule tagged with specific antibodies for the lateral flow test, or a substrate for a specific colorimetric dye). In this way, the target sequence in the sample is indirectly detected via the reporter molecule.

In some embodiments, the detection region 113 is configured to perform a CRISPR/Cas analysis in which the guide nucleic acid (e.g., gRNA) is incubated with the programmable nuclease to form ribonucleoprotein (RNP) complexes prior to incubation with the amplified sample. As noted above, the guide nucleic acid comprises a segment that has reverse complementarity to a segment of the target nucleic acid sequence. The programmable nuclease, in some embodiments, is capable of sequence-independent cleavage after the guide nucleic acid binds to its specific target sequence.

In some embodiments, the detection region 113 is configured to perform a CRISPR/Cas analysis in which the programmable nuclease is activated upon binding of the complex of the bound gRNA and target nucleic acid and initiates trans cleavage activity of an RNA reporter by RNA or DNA. In some embodiments, the programmable nuclease binds to a target RNA to initiate trans cleavage of an RNA reporter. Such programmable nucleases are referred to as an RNA-activated programmable RNA nuclease. In some embodiments, the programmable nuclease binds to a target DNA to initiate trans cleavage of an RNA reporter; this programmable nuclease is referred to as a DNA-activated programmable RNA nuclease. In some embodiments, the programmable nuclease may be activated by a target RNA or a target DNA. For example, a Cas13 programmable nuclease, such as Cas13a is activated by a target RNA nucleic acid or a target DNA nucleic acid to transcollaterally cleave RNA reporter molecules. In some embodiments, the Cas13 binds to a target single stranded DNA (ssDNA), initiating trans cleavage of RNA reporters. In other embodiments, the programmable nuclease binds to a target DNA to initiate trans cleavage of a DNA reporter. This programmable nuclease is referred to as a DNA-activated programmable DNA nuclease.

In some embodiments, the programmable nuclease is a Cas protein, such as Cas9, Cas12a, Cas12b, Cas13, or Cas14. Cas9 and Cas12 nucleases are DNA-specific, while Cas13 is RNA-specific. Cas14 targets single-stranded DNA.

As noted above, the programmable nuclease may be activated upon binding of a guide nucleic acid with a target nucleic acid, such that the activated programmable nuclease can then cleave the target nucleic acid, and may have trans cleavage activity. Trans cleavage activity, in some embodiments, is non-specific cleavage of nearby single-stranded nucleic acids by the activated programmable nuclease. For example, the activated programmable nuclease may cleave a detector nucleic acid comprising a detection moiety. The resulting nucleic acid with detection moiety may be released or separated from the reporter, generating a detectable signal. The detection moiety, in some embodiments, is a fluorophore, a dye, a polypeptide or a nucleic acid. In some embodiments, the detection moiety binds to a capture molecule (e.g., capture antibody) on a lateral flow strip, as described herein.

Guide nucleic acids bind to the single stranded target nucleic acid comprising a portion of a nucleic acid from a pathogen described herein. Therefore, in some embodiments, the guide nucleic acid is complementary to the target nucleic acid. In some embodiments, the guide nucleic acid binds specifically to the target nucleic acid. The target nucleic acid may be a RNA, DNA, or synthetic nucleic acids. In some embodiments, a guide nucleic acid comprises a crRNA and tracrRNA. The guide RNA may not be naturally occurring and may be made by artificial combination of otherwise separate segments of sequence. For example, in some embodiments, the artificial combination is performed by chemical synthesis, by genetic engineering techniques, or by the artificial manipulation of isolated segments of nucleic acids. The target nucleic acid can be designed to provide desired functions. In some embodiments, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length, and in some embodiments, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some embodiments, the targeting region of a guide nucleic acid has a length that ranges from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some embodiments, the targeting region of a guide nucleic acid has a length ranging from about 10 nt to about 60 nt, from about 20 nt to about 50 nt, or from about 30 nt to about 40 nt. In some embodiments, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt.

In some embodiments, the guide nucleic acid may be selected from a group of guide nucleic acids that have been screened against the nucleic acid of a strain of an infection or genomic locus of interest. The guide nucleic acid, in some embodiments, may be selected from a group of guide nucleic acids that have been screened against the nucleic acid of a strain of SARS-CoV-2. In some embodiments, guide nucleic acids that are screened against the nucleic acid of a target sequence of interest can be pooled. Without wishing to be bound by theory, it is thought that pooled guide nucleic acids directed against a single target nucleic acid can ensure broad coverage of the target nucleic acid within a single reaction. The pooled guide nucleic acids, in some embodiments, are directed to different regions of the target nucleic acid and may be sequential or non-sequential. In some embodiments the method for detecting a target nucleic acid comprises contacting a target nucleic acid (e.g., an amplified nucleic acid sample, as described herein) to a pool of guide nucleic acids and a programmable nuclease, wherein a guide nucleic acid of the pool of guide nucleic acids has a sequence selected from a group of guide nucleic acids that have reverse complementarity to a sequence of the target nucleic acid; and then assaying for a signal produced by cleavage of at least some detector nucleic acids of a population of detector nucleic acids (e.g., using a lateral flow strip or colorimetric assay described herein.

In some embodiments, the detection region 113 is configured to perform a CRISPR/Cas analysis that detects more than one target sequence, such as 2, 3, 4, 5, 6, 7, 8, 9, 10, or more target sequences. In these embodiments, the amplified nucleic acid sample is contacted with a group of guide nucleic acid sequences, wherein the group comprises guide nucleic acids that separately target each of the target sequences. In such embodiments, each guide nucleic acid may be associated with a different programmable nuclease. Binding of the guide nucleic acid to its target will lead to activation of a specific programmable nuclease. In some embodiments, each guide nucleic acid has a specific programmable nuclease (e.g., if the assay is screening for two different target nucleic acids, there will be at least two different guide nucleic acids, and at least two different programmable nucleases, each of which is specific for the unique guide nucleic acid). The programmable nuclease become activated and releases a unique detectable moiety if the target sequence is present. If two target sequences are assayed, then at least two different programmable nucleases will be used, and their respective activation will results in the release of two unique detection moieties. In this way, an operator can determine if 0, 1, or 2 target sequences are present in the sample.

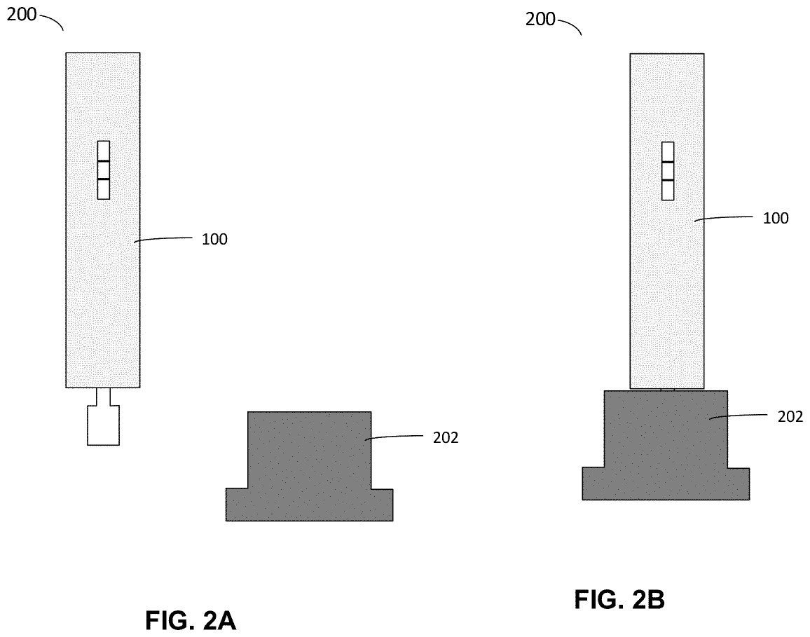

FIGS. 2A-2B depict a system comprising a diagnostic device and a container containing a buffer, according to some embodiments. In the example of FIGS. 2A-2B, diagnostic device 100 shown in FIGS. 1A-1B is depicted as part of a system with container 202. The container contains a buffer and the diagnostic device 100 may be inserted into the container so that the buffer solution is absorbed by the swab of the diagnostic device. The buffer solution may thereby aid (or may cause) a sample on the swab to flow along the lateral flow strip of the diagnostic device, where it may be processed and analyzed as discussed above.

According to some embodiments, the container 202 may comprise a rehydration buffer. The rehydration buffer, in some embodiments, comprises Tris pH 8.0, poly(ethylene glycol), magnesium acetate tetrahyrate, potassium acetate, and nuclease free water. As an example, the rehydration buffer may comprise: 25 mM Tris buffer, 5% (w/v) poly(ethylene glycol) 35,000 kDa, 14 mM magnesium acetate tetrahydrate, 100 mM potassium acetate, and >85% volume nuclease free water.

In some embodiments, the container 202 may comprise a seal, such as a foil seal, over the buffer within the container. As such, the buffer may be sealed inside the container until the seal is punctured or removed by a user. In some embodiments, the diagnostic device 100 may be configured to puncture the seal when it is inserted into the container. The housing of the diagnostic device may, for example, include a structure suitable to puncture the seal, or the swab itself may be shaped to puncture the seal.

According to some embodiments, the container 202 may include one or more heating elements. A heating element may be arranged proximate to the buffer to heat the buffer solution. For instance, some amplification reactions may require, or may be improved with, the liquid within the strip being raised above room temperature. A suitable heating element may include a resistive heater. In some embodiments, the heater may be a USB-powered heat source. According to some embodiments, a heating element arranged within the container 202 may be configured to heat the buffer to at least 35° C., 40° C., 45° C., 50° C., 55° C., 60° C., 65° C., 70° C., 75° C., 80° C., 85° C., 90° C., or 95° C.

According to some embodiments, the container may include one or more locking mechanisms configured to mate with the diagnostic device 100 when it is inserted into the container (or subsequent to insertion). For instance, the diagnostic device and container may contain corresponding snap-fit components (e.g., the diagnostic device may include a recess and the container may include a corresponding cantilever to snap-fit into the recess). A snap-fit connection may be temporary or permanent. A permanent connection may aid in ensuring that a user correctly performs a test using system 200.

According to some embodiments, the exterior of the container 202 may include one or more fiducial targets. A portable computing device (e.g., a mobile phone or tablet) may identify the one or more fiducial targets in conjunction with fiducial target(s) on the housing of the diagnostic device to determine whether or not the diagnostic device is properly inserted into the container, based on a known spatial relationship between the fiducial target(s) on the container and the fiducial target(s) on the diagnostic device. Such a process may provide a further check that a test using system 200 is being properly performed.

According to some embodiments, system 200 may be formulated as a kit (or part of a kit). As used herein a “kit” comprises a package or an assembly including at least the container 202 and the diagnostic device 100. A kit may also comprise any number of sampling devices (e.g., swabs), heaters, reaction tubes, wells, chambers, or other vessels. Each of the components of the kit (e.g., reagents) may be provided in liquid form (e.g., in solution) or in solid form (e.g., a dried powder, lyophilized).

A kit may, in some cases, include instructions for performing any one of the tests described herein. The instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications). In some embodiments, the instructions are provided as part of a software-based application, as described herein.

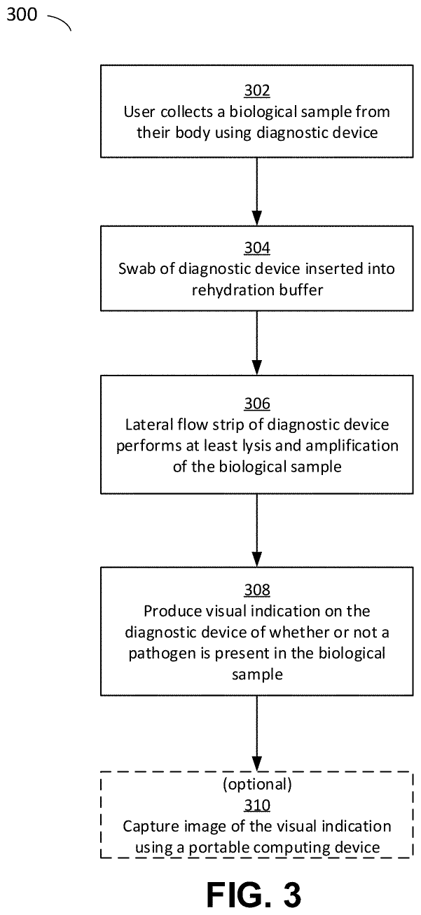

FIG. 3 is a flowchart of a method of performing a diagnostic test for one or more pathogens using a self-administrable nucleic acid test, according to some embodiments. Method 300 may be performed by a user with the components of system 200 shown in FIGS. 2A-2B—that is, the diagnostic device 100 and the container 202. In this manner, the method 300 represents a self-administered test.

In act 302, the user collects a biological sample from their body with the swab of the diagnostic device. Illustrative samples may include body fluids (e.g. blood, serum, plasma, sputum, urine, tear fluid, or feces), tissue extracts, culture media (e.g., a liquid in which a cell, such as a pathogen cell, has been grown), environmental samples, agricultural products or other foodstuffs, and their extracts.

In some embodiments, act 302 may comprise the user colleting a mucus (e.g., nasal secretion) sample, and/or a saliva (spit) sample. In some embodiments, the user self-collects an anterior nares sample by swabbing either or both nostrils (e.g., for 10 seconds). In some embodiments, when the sample is a saliva sample, the saliva sample may be 1 mL-4 mL or more. In some embodiments, the saliva sample is 1 mL, 1.5 mL, 2 mL, 2.5 mL, 3, mL, 3.5 mL, 4 mL, or more. Saliva has been found to have a mean concentration of SARS-Cov-2 RNA of 5 fM (Kai-Wang To et al., 2020), an amount that is detectable by at least one of the tests described herein. In some embodiments, the concentration of a target nucleic acid molecule (e.g., SARS-CoV-2 RNA) in the sample is present at a concentration of 5 aM, 10 aM, 15 aM, 20 aM, 25 aM, 30 aM, 35 aM, 40 aM, 50 aM, 75 aM, 100 aM, 150 aM, 200 aM, 300 aM, 400 aM, 500 aM, 600 aM, 700 aM, 800 aM, 900 aM, 1 fM, 5 fM, 10 fM, 15 fM, 20 fM, 25 fM, 30 fM, 35 fM, 40 fM, 50 fM, 75 fM, 100 fM, 150 fM, 200 fM, 300 fM, 400 fM, 500 fM, 600 fM, 700 fM, 800 fM, 900 fM, 1 pM, 5 pM, 10 pM, or more. In some embodiments, in act 302 the user collects a cell scraping, such as from the mouth or interior cheek.

The user performing act 302 may be a subject who is suspected of having the disease or diseases the test screens for, such as a coronavirus (e.g., COVID-19) or influenza (e.g., influenza type A or influenza type B). Other indications, as described herein, are also envisioned. The user may be asymptomatic, or may present with one or more symptoms of the disease(s). Symptoms of coronaviruses (e.g., COVID-19) include, but are not limited to, fever, cough (e.g., dry cough), generalized fatigue, sore throat, headache, loss of taste or smell, runny nose, nasal congestion, muscle aches, and difficulty breathing (shortness of breath). For influenza, the symptoms include, but are not limited to, fever, chills, muscle aches, cough, congestion, runny nose, headaches, and generalized fatigue. In some embodiments, The user is asymptomatic, but has had contact within the past 14 days with a person that has tested positive for the virus.

In some embodiments, the sample collected in act 302 comprises a target nucleic acid sequence (molecule). Target nucleic acid molecules may comprise double-stranded and single-stranded nucleic acid molecules (e.g., DNA, RNA, and other nucleobase polymers known in the art capable of hybridizing with a nucleic acid molecule described herein). RNA molecules suitable for detection with any of the tests or methods described herein include, but are not limited to, double-stranded and single-stranded RNA molecules that comprise a target sequence (e.g., messenger RNA, viral RNA, ribosomal RNA, transfer RNA, microRNA and microRNA precursors, and siRNAs or other RNAs described herein or known in the art). DNA molecules suitable for detection with any one of the tests or methods described herein include, but are not limited to, double stranded DNA (e.g., genomic DNA, plasmid DNA, mitochondrial DNA, viral DNA, and synthetic double stranded DNA). Single-stranded DNA target nucleic acid molecules include, for example, viral DNA, cDNA, and synthetic single-stranded DNA, or other types of DNA known in the art.

In act 304, at least part of the diagnostic device is inserted into the container such that the sample collected in act 302 is submerged in a buffer within the container. As described above, saturation of the swab of the diagnostic device may cause flow of fluid that includes at least some of the sample along the lateral flow strip of the diagnostic device, thereby initiating a nucleic acid test as described. Act 304 may include puncturing a seal over the buffer within the container, and may include causing the diagnostic device to attach or lock to the container, both as described above.

In act 306, the lateral flow strip of the diagnostic device performs at least lysis of the sample and amplification of resulting material, various embodiments of which are described above. In act 308, one or more visual indications of the result of the test are produced on or within the diagnostic device that indicate whether or not a particular pathogen was present in the sample provided. As discussed above, a number of such indications may be produced on the same device and may produce indications for different pathogens.

Method 300 may end with act 308, or optionally may include act 310 in which an image of the one or more visual indications produced in act 306 are captured by a portable computing device. As discussed above, any results of the test performed by the diagnostic device may be captured by a portable computing device and conveyed to the user by the portable computing device in easily understood terms rather than requiring the user to determine the result(s) based on the presence or absence of visual indications on the diagnostic device.

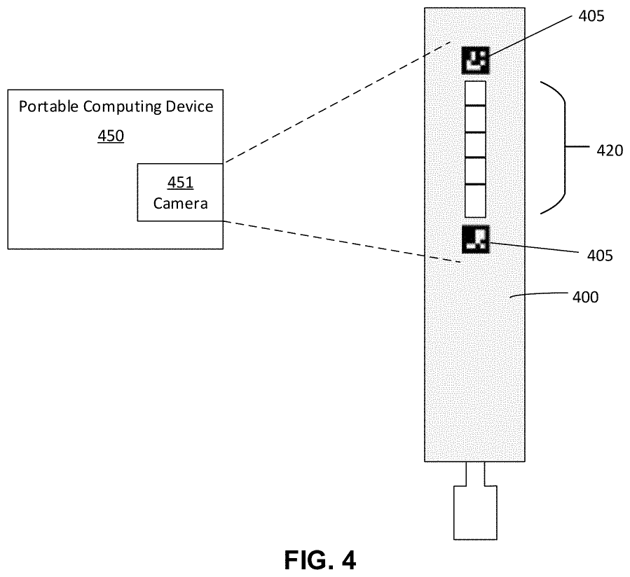

Act 308 may be illustrated by FIG. 4, according to some embodiments. As shown in the example of FIG. 4, a portable computing device 450 comprising a camera 451 is operated to capture an image of at least part of the diagnostic device 400. In the example of FIG. 4, the diagnostic device 400 includes fiducial markers 405 that allow the portable computing device 450 to determine, based on a captured image that includes the fiducial markers 405, where in the image the various test and control lines are located in window 420. At least one processor of the portable computing device 450 may thereby be configured to capture an image using the camera and an image sensor, and to analyze the image to detect the position and/or orientation of expected fiducial markers 405. Based on the detected fiducial markers and information about the relative spatial position of the fiducial markers and the test line(s) and control line(s) on the diagnostic device, the at least one processor may determine, for each of the test lines and control lines of the diagnostic device, which test line(s) and control line(s) are present, and which are absent. The at least one processor may then make a determination of whether or not a particular pathogen was present in a sample, and communicate the result of this determination to a user of the portable computing device.

For instance, the user may take a picture of at least part of the diagnostic device that includes the window 420 and the fiducial targets 405 with their smartphone that is running a mobile application. After capturing the image, a computer vision algorithm is executed by the smartphone to electronically identify the bands. In some embodiments, the user may be asked to manually enter a band pattern result by examining the diagnostic device. In such cases, if the band-pattern result determined by the algorithm differs from the band pattern result entered by the user, the user is asked to double-check that they entered the correct band-pattern, and the user is given the opportunity to re-enter the band pattern. Alternatively, the band pattern may be determined solely by the smartphone. In either case, once a band pattern is determined, the user may be shown a “Test Complete” screen in the mobile application, which tells the user if the test result is positive, negative, or invalid. In addition to providing the test result, careful language is used to ensure that the user can properly interpret the meaning of the result.

According to some embodiments, the portable computing device 450 may be configured to communicate with a remote server having a database to store test results. In some embodiments, the database also stores patient information and the portable computing device may transmit information identifying the user performing the test (or the user from whom the sample being tested was obtained) to the database, and the database may store the test results associated with the user information. In some embodiments, the user/patient information is an electronic medical record. In some embodiments, the patient information includes at least one of name, social security number, date of birth, address, phone number, email address, medical history, and medications. The system may also include user or patient tracking capabilities, such as with use of smartphones or remote devices with tracking capabilities. These locations can be tracked and monitored by the remote server, and assess and notify others who come into contact or within a certain distance of any user, such as a user who has tested positive for a viral illness. The locations may also be communicated to a central database server and/or to a remote doctor or other.

According to some embodiments, the portable computing device 450 may be configured to communicate this information (e.g., an image of the resultant lateral flow test strip) to a secure, HIPAA-compliant, cloud-based software infrastructure. This software infrastructure then facilitates simple, fast, and scalable reporting to the federal and state health agencies.

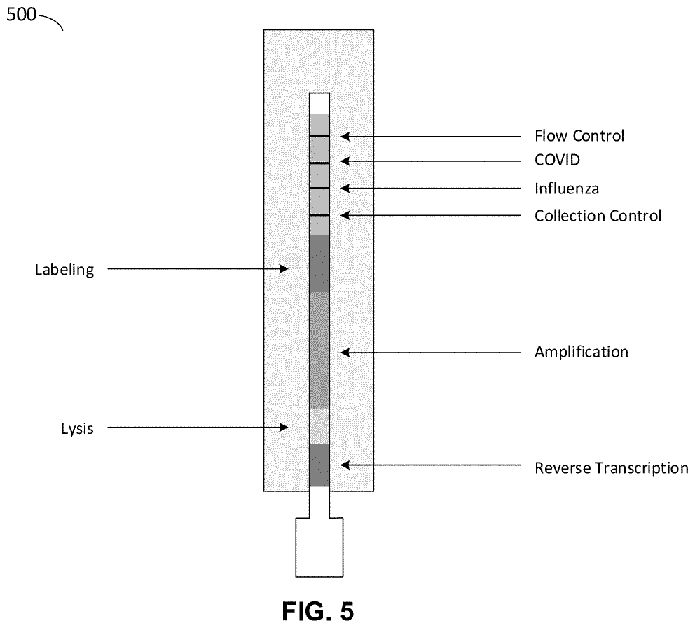

FIG. 5 depicts a cross-sectional view of a diagnostic device for performing a self-administrable nucleic acid test, according to some embodiments. Diagnostic device 500 is depicted for illustrative purposes and depicts an example of diagnostic device 100 shown in FIG. 1 that includes two test lines (for COVID and influenza) and two control lines (one for ensuring that liquid flow has reached the end of the lateral flow strip, and another to confirm that nucleic acids from a subject were analyzed by the device). Each of these test and control lines are described above in relation to FIG. 1. In addition, illustrative diagnostic device 500 includes a reverse transcription region and a labeling region on its lateral flow strip. These optional regions of the lateral flow strip are also described above in relation to FIG. 1.

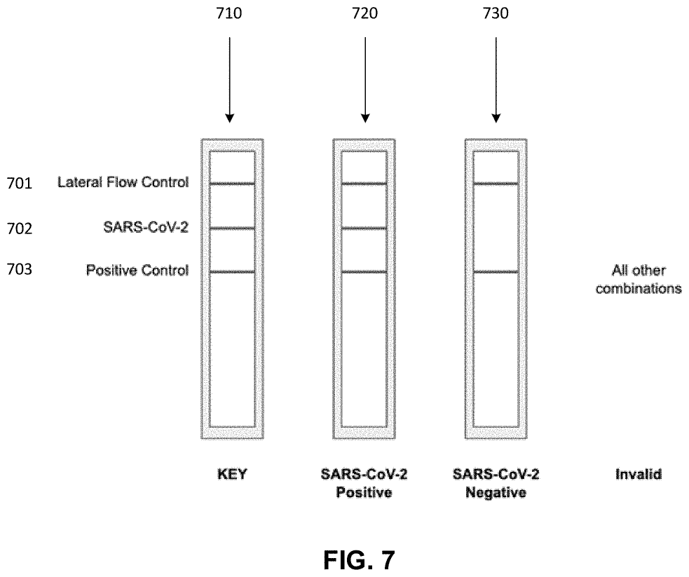

FIG. 7 depicts an illustrative lateral flow strip key for interpreting results, according to some embodiments. As shown in FIG. 7, a detection region of a lateral flow strip may include three lines—a flow control line 701, a test line for SARS-CoV-2 702, and a positive control line 703. Since both the control lines 701 and 703 are necessary for a completed test, the only valid combinations that may be produced from lines shown in the key 710 are shown as positive and negative tests 720 and 730, respectively. Any other combinations of lines being present or absent would represent an incomplete test as either the flow of material did not reach control line 701, or an expected nucleic acid (e.g., RNaseP for a human sample) was not detected by control line 703.

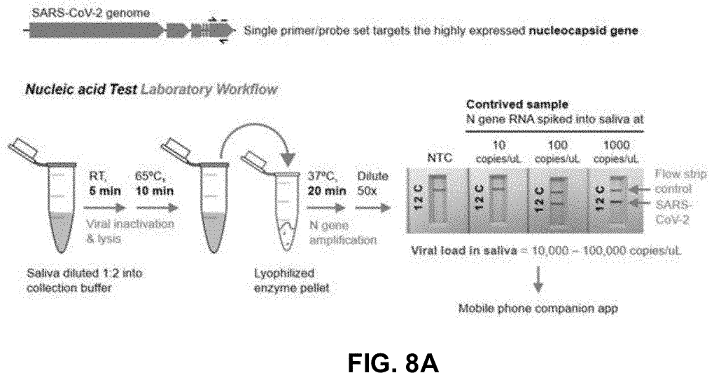

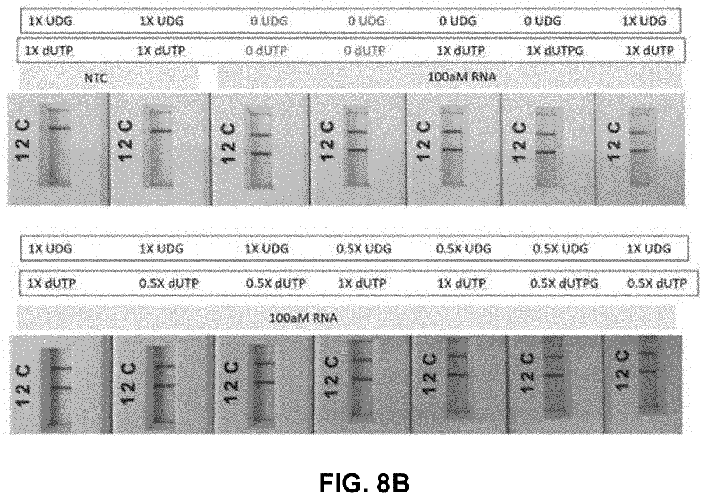

FIG. 8A depicts a schematic illustrating a test laboratory workflow, according to some embodiments. According to some embodiments, an experiment was undertaken to determine how sensitive the lateral flow strip tests described herein are with respect to different concentrations of viral load. As shown in FIG. 8A, a single primer/probe set targeting the SARS-Cov-2 nucleocapsid gene was used. Contrived saliva samples (spiked with nucleocapsid gene RNA at the 10, 100, or 1000 copies per μL) were diluted 1:2 in collection buffer. The samples were incubated at room temperature for five minutes, and then heated to 65° C. for 10 minutes for inactivation and lysis. The resulting solution was added to a new tube comprising a lyophilized enzyme pellet comprising amplification enzymes. The pellet was dissolved in the solution, and the tube as heated to 37C for 20 minutes to amplify the nucleocapsid gene DNA. Samples were then diluted 50-fold and run on lateral flow test strips. As shown in FIG. 8A, viral loads as low as 100 copies/μL were detected.

Further, different concentrations of UDG and dUTP were examined. As shown in FIG. 8B, all of the combinations tested (0 UDG, 0.2×UDG, 1 UDG, 0 dUTP, 0.5× dUTP, 1 dTUP) showed the SARS-CoV-2 line. Concentrations of 100 aM RNA were used.

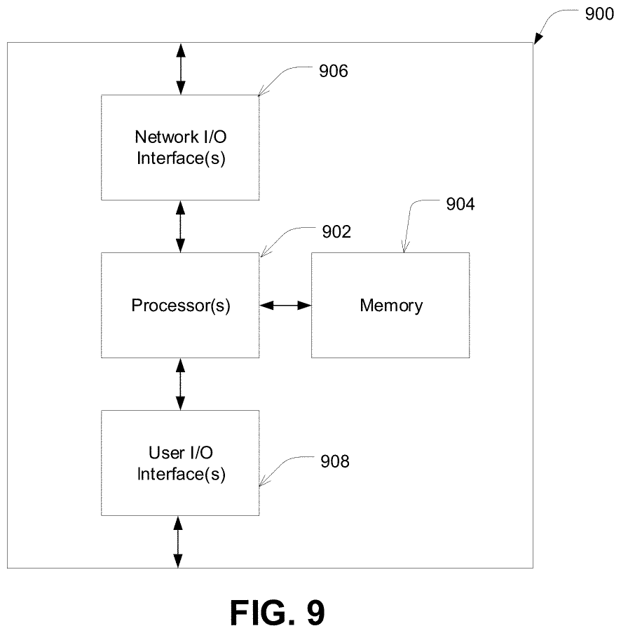

In some embodiments, systems and techniques described herein may be implemented using one or more computing devices. In particular, a portable computing device may be operated to detect a result on a diagnostic device as described above. Embodiments are not, however, limited to operating with any particular type of computing device. By way of further illustration, FIG. 9 is a block diagram of an illustrative computing device 900. Computing device 900 may include one or more processors 902 and one or more tangible, non-transitory computer-readable storage media (e.g., memory 904). Memory 904 may store, in a tangible non-transitory computer-recordable medium, computer program instructions that, when executed, implement any of the above-described functionality. Processor(s) 902 may be coupled to memory 904 and may execute such computer program instructions to cause the functionality to be realized and performed.

Computing device 900 may also include a network input/output (I/O) interface 906 via which the computing device may communicate with other computing devices (e.g., over a network), and may also include one or more user I/O interfaces 908, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. As an example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A diagnostic device for detecting a disease, the diagnostic device comprising:
a housing;
a lateral flow strip integrated with the housing including:
a lysis region comprising at least one lysis buffer;
an amplification region comprising one or more lyophilized amplification reagents; and
a detection region configured to produce an indication of whether or not the disease was detected; and
a swab coupled to the lateral flow strip and arranged outside the housing.

2. The diagnostic device of claim 1, wherein the housing is formed from a thin film sealed around the lateral flow strip.

3. The diagnostic device of claim 1, wherein the housing comprise first and second housing elements joined together and arranged on opposing sides of the lateral flow strip.

4. (canceled)

5. The diagnostic device of claim 1, wherein the lysis region of the lateral flow strip further comprises lyophilized lysis enzymes and detergents.

6. The diagnostic device of claim 1, wherein the one or more lyophilized amplification reagents include one or more reagents for isothermal amplification.

7. (canceled)

8. The diagnostic device of claim 1, wherein the housing comprises a plurality of fiducial markers disposed on an exterior surface.

9. The diagnostic device of claim 1, wherein the housing comprises a readout window through which at least a portion of the lateral flow strip is visible.

10-12. (canceled)

13. The diagnostic device of claim 1, wherein the swab is fluidically coupled to the lateral flow strip such that fluid absorbed by the swab flows into the lateral flow strip via capillary action.

14. (canceled)

15. The diagnostic device of claim 1, wherein the detection region comprises a human sample control line and a first disease test line, wherein the first disease test line is configured to detect the presence of a coronavirus, and wherein the coronavirus is SARS-CoV-2.

16-38. (canceled)

39. The diagnostic device of claim 1, further comprising a heater arranged within the housing adjacent to at least a portion of the lateral flow strip.

40. A system for performing a self-administrable test to detect a disease, the system comprising:
a diagnostic device comprising:
a housing;
a lateral flow strip integrated with the housing including:
a lysis region comprising at least one lysis buffer; and
an amplification region comprising one or more lyophilized amplification reagents; and
a swab coupled to the lateral flow strip and arranged outside the housing; and
a container comprising a rehydration buffer and a heater configured to heat the rehydration buffer, wherein the container is configured to be coupled to the diagnostic device such that the swab is arranged within the rehydration buffer.

41. (canceled)

42. The system of claim 40, wherein the container comprises a foil seal over the rehydration buffer, and wherein the swab of the diagnostic device is configured to puncture the foil seal of the container.

43-50. (canceled)

51. The system of claim 40, wherein the housing comprises a plurality of fiducial markers disposed on an exterior surface.

52. The system of claim 40, wherein the housing comprises a readout window through which at least a portion of the lateral flow strip is visible.

53-59. (canceled)

60. A method of detecting a pathogen, the method comprising:
collecting a biological sample from a first user's body, wherein a second user manipulates a diagnostic device to collect the biological sample from the first user's body, the diagnostic device comprising a lateral flow strip including a lysis region comprising at least one lysis buffer and an amplification region comprising one or more lyophilized amplification reagents, and a swab coupled to the lateral flow strip, wherein the biological sample is collected on the swab; and
performing, by the lateral flow strip of the diagnostic device:
lysing of at least some of the biological sample;
amplification of nucleic acids from the biological sample;
screening of the amplified nucleic acids for the pathogen; and
producing a visual indication of whether or not the pathogen is present in the biological sample.

61. The method of claim 60, wherein first user and the second user are the same user such that the first user collects the biological sample from their own body.

62. The method of claim 60, wherein the pathogen includes a viral disease comprising at least one of COVID-19, influenza A, and influenza B.

63. (canceled)

64. The method of claim 60, wherein lysis of the biological sample comprises immersing the swab in a rehydration buffer and heating the biological sample and buffer to at least 55° C.

65. The method of claim 60, further comprising detecting the visual indication using a portable computing device.

66. The method of claim 65, wherein detecting the visual indication comprises identifying, using at least one processor of the portable computing device, at least one fiducial mark disposed on the diagnostic device within an image of the diagnostic device.

67-68. (canceled)


SEQUENCE LISTING (47 sequences)

SEQ ID NO: 1 - DNA, 30 bp, Artificial Sequence
  Synthetic
tctgataatggaccccaaaatcagcgaaat

SEQ ID NO: 2 - DNA, 30 bp, Artificial Sequence
  Synthetic
ctccattctggttactgccagttgaatctg

SEQ ID NO: 3 - DNA, 30 bp, Artificial Sequence
  Synthetic
gcaactgagggagccttgaatacaccaaaa

SEQ ID NO: 4 - DNA, 30 bp, Artificial Sequence
  Synthetic
tgaggaagttgtagcacgattgcagcattg

SEQ ID NO: 5 - DNA, 30 bp, Artificial Sequence
  Synthetic
aaggaactgattacaaacattggccgcaaa

SEQ ID NO: 6 - DNA, 30 bp, Artificial Sequence
  Synthetic
ttccatgccaatgcgcgacattccgaagaa

SEQ ID NO: 7 - DNA, 30 bp, Artificial Sequence
  Synthetic
aaattttggggaccaggaactaatcagaca

SEQ ID NO: 8 - DNA, 30 bp, Artificial Sequence
  Synthetic
tggcacctgtgtaggtcaaccacgttcccg

SEQ ID NO: 9 - DNA, 32 bp, Artificial Sequence
  Synthetic
accccaaaatcagcgaaatgcaccccgcatta

SEQ ID NO: 10 - DNA, 25 bp, Artificial Sequence
  Synthetic
gtagaaataccatcttggactgaga

SEQ ID NO: 11 - DNA, 28 bp, Artificial Sequence
  Synthetic
gtctgataatggaccccaaaatcagcga

SEQ ID NO: 12 - DNA, 32 bp, Artificial Sequence
  Synthetic
tagtagaaataccatcttggactgagatcttt

SEQ ID NO: 13 - DNA, 45 bp, Artificial Sequence
  Synthetic
agaatggagaacgcagtggggcgcgatcaaaacaacgtcggcccc

SEQ ID NO: 14 - DNA, 29 bp, Artificial Sequence
  Synthetic
agaatggagaacgcagtggggcgcgatca

SEQ ID NO: 15 - DNA, 50 bp, Artificial Sequence
  Synthetic
cagtaaccagaatggagaacgcagtggggcgcgatcaaaacaacgtcggc

SEQ ID NO: 16 - DNA, 37 bp, Artificial Sequence
  Synthetic
cagtaaccagaatggagaacgcagtggggcgcgatca

SEQ ID NO: 17 - DNA, 30 bp, Artificial Sequence
  Synthetic
gtactgccactaaagcatacaatgtaacac

SEQ ID NO: 18 - DNA, 31 bp, Artificial Sequence
  Synthetic
aatatgcttattcagcaaaatgacttgatct

SEQ ID NO: 19 - DNA, 33 bp, Artificial Sequence
  Synthetic
cagacaaggaactgattacaaacattggccgca

SEQ ID NO: 20 - DNA, 18 bp, Artificial Sequence
  Synthetic
cggtggacaaattgtcac

SEQ ID NO: 21 - DNA, 22 bp, Artificial Sequence
  Synthetic
cttctctggatttaacacactt

SEQ ID NO: 22 - DNA, 28 bp, Artificial Sequence
  Synthetic
ttacaagcttaaagaatgtctgaacact

SEQ ID NO: 23 - DNA, 27 bp, Artificial Sequence
  Synthetic
ttgaatttaggtgaaacatttgtcacg

SEQ ID NO: 24 - DNA, 47 bp, Artificial Sequence
  Synthetic
tcagcacacaaagccaaaaatttatctgtgcaaaggaaattaaggag

SEQ ID NO: 25 - DNA, 45 bp, Artificial Sequence
  Synthetic
tattggtggagctaaacttaaagccctgtacaatccctttgagtg

SEQ ID NO: 26 - DNA, 51 bp, Artificial Sequence
  Synthetic
tcagcacacaaagccaaaaatttatttttctgtgcaaaggaaattaaggag

SEQ ID NO: 27 - DNA, 49 bp, Artificial Sequence
  Synthetic
tattggtggagctaaacttaaagccttttctgtacaatccctttgagtg

SEQ ID NO: 28 - DNA, 18 bp, Artificial Sequence
  Synthetic
tgcttcagtcagctgatg

SEQ ID NO: 29 - DNA, 22 bp, Artificial Sequence
  Synthetic
ttaaattgtcatcttcgtcctt

SEQ ID NO: 30 - DNA, 41 bp, Artificial Sequence
  Synthetic
tcagtactagtgcctgtgcccacaatcgtttttaaacgggt

SEQ ID NO: 31 - DNA, 43 bp, Artificial Sequence
  Synthetic
tcgtatacagggcttttgacatctatcttggaagcgacaacaa

SEQ ID NO: 32 - DNA, 17 bp, Artificial Sequence
  Synthetic
ctgcacttacaccgcaa

SEQ ID NO: 33 - DNA, 22 bp, Artificial Sequence
  Synthetic
gtagctggttttgctaaattcc

SEQ ID NO: 34 - DNA, 17 bp, Artificial Sequence
  Synthetic
ttgatgagctggagcca

SEQ ID NO: 35 - DNA, 18 bp, Artificial Sequence
  Synthetic
caccctcaatgcagagtc

SEQ ID NO: 36 - DNA, 41 bp, Artificial Sequence
  Synthetic
gtgtgaccctgaagactcggttttagccactgactcggatc

SEQ ID NO: 37 - DNA, 45 bp, Artificial Sequence
  Synthetic
cctccgtgatatggctcttcgtttttttcttacatggctctggtc

SEQ ID NO: 38 - DNA, 20 bp, Artificial Sequence
  Synthetic
atgtggatggctgagttgtt

SEQ ID NO: 39 - DNA, 20 bp, Artificial Sequence
  Synthetic
catgctgagtactggacctc

SEQ ID NO: 40 - DNA, 13 bp, Artificial Sequence
  Synthetic
cagccatccacat

SEQ ID NO: 41 - protein, 8 aa, Artificial Sequence
  Synthetic
DYKDDDDK

SEQ ID NO: 42 - protein, 6 aa, Artificial Sequence
  Synthetic
HHHHHH

SEQ ID NO: 43 - protein, 9 aa, Artificial Sequence
  Synthetic
YPYDVPDYA

SEQ ID NO: 44 - protein, 10 aa, Artificial Sequence
  Synthetic
EQKLISEEDL

SEQ ID NO: 45 - DNA, 15 bp, Artificial Sequence
  Synthetic
aacaacgtcggcccc

SEQ ID NO: 46 - DNA, 13 bp, Artificial Sequence
  Synthetic
aacaacgtcggcc

SEQ ID NO: 47 - DNA, 15 bp, Artificial Sequence
  Synthetic
attgcacaatttgcc